(19) United States
(12) Patent Application Publication
(10) Pub. No.: US 2024/0270942 A1
(43) Pub. Date: Aug. 15, 2024

(54) 3D-PRINTABLE SHEAR-THINNING POLYSACCHARIDE-BASED NANOCOMPOSITE HYDROGEL FOR BIOMIMETIC TISSUE ENGINEERING

(71) Applicant: UNIVERSITY OF FLORIDA RESEARCH FOUNDATION, INCORPORATED, Gainesville, FL (US)

(72) Inventor: Mei HE, Gainesville, FL (US)

(21) Appl. No.: 18/568,137

(22) PCT Filed: Jun. 17, 2022
(86) PCT No.: PCT/US22/33966

§ 371 Date: Dec. 7, 2023

Related U.S. Application Data
(60) Provisional application No. 63/212,474, filed on Jun. 18, 2021.

(51) Int. Cl. C08L 3/02; A61L 26/00; B33Y 70/00

(52) U.S. Cl. CPC C08L 3/02; A61L 26/0023; A61L 26/0033; A61L 26/0038; A61L 26/008; B33Y 70/00; A61L 2400/12

(57) ABSTRACT

Described herein are biocompatible nanocomposite hydrogel compositions for three-dimensional printing, comprising: collagen, starch, and gelatin nanoparticles. Additionally described are methods of using three-dimensional objects printed from the a nanocomposite hydrogel compositions for healing wounds and regenerating tissue.

### CROSS REFERENCE TO RELATED APPLICATIONS

This application claims the benefit of priority to U.S. Provisional Patent Application No. 63/212,474, filed Jun. 18, 2021, which is incorporated by reference herein in its entirety for all purposes.

### GOVERNMENT SUPPORT

This invention was made with government support under R35 GM133794 awarded by the National Institutes of Health. The government has certain rights in the invention.

### FIELD OF THE INVENTION

The presently disclosed subject matter relates generally to biocompatible nanocomposite hydrogel compositions for three-dimensional printing, which can be used for mimicking in vivo tissue microenvironment, would healing, tissue repair, and transplantation.

### BACKGROUND

3D bioprinting offers great potential for creating stand-alone and well-defined tissue architectures with intricate patterns or customized zones (Askari et al., 2021; Liu et al., 2016; Zhuang et al., 2020). A 3D printed scaffold can greatly improve mass transport throughout the cultured tissue system (Attalla et al., 2018; Idaszek et al., 2019), which is attractive for tissue engineering applications. One of the determining factors for successful printing is the selection of the bio-ink (Unagolla & Jayasuriya, 2020; Zhuang et al., 2018). The ideal 3D bio-ink should fulfill the requirements of the adopted printing technique, for instance, viscosity, shear-thinning, low yield stress, and thixotropy. Also important is the ability of the bio-ink to support the attachment and 3D growth of seeded cells (Schwab et al., 2020; Y. S. Zhang & Khademhosseini, 2017). Unfortunately, scientists have encountered challenges designing materials with desired mechanical properties that can maintain their biological function. The subject matter described herein addresses this problem.

### BRIEF SUMMARY

In one aspect, the presently disclosed subject matter is directed to a nanocomposite hydrogel composition for three-dimensional printing, comprising: collagen, starch, and gelatin nanoparticles.

In another aspect, the presently disclosed subject matter is directed to a three-dimensional object printed from the nanocomposite hydrogel compositions described herein, wherein said three-dimensional object is structurally integrous and stable.

In another aspect, the presently disclosed subject matter is directed to a method of treating a wound and/or regenerating tissue in a subject in need thereof, comprising administering to a treatment site in said subject the nanocomposite hydrogel compositions described herein.

In another aspect, the presently disclosed subject matter is directed to a method of preparing the nanocomposite hydrogel compositions described herein.

These and other aspects are described herein.

### BRIEF DESCRIPTION OF THE DRAWINGS

FIG. 1 shows an assessment of gelatin nanoparticles (GNPs) in terms of morphology, size distribution, and toxicity on cell proliferation. a) Size and concentration measurements using Nanoparticle Tracking Analysis (NTA). The standard deviation is indicated by the shading surrounding the line b) FESEM images of gelatin nanoparticles in DI water with lower and higher magnifications (scale bar: 400 nm and 50 nm, as indicated in the inset). c) Effects of GNPs with varied concentration on cell proliferation over a 5-day culture, determined using a Prestoblue assay (n=9 technical replicates). From left-to-right in the bar graph, each bar represents a GNP concentration of 0, 10², 104, 10⁶, 10⁸, and 10¹⁰ (each in particles/mL).

FIG. 2 shows a plot of the size distribution analysis of GNPs using dynamic light scattering. The standard deviation is indicated by the shaded coloring surrounding the dotted black line.

FIG. 3 shows a schematic of the development of 3D printable nanoparticle/hydrogel composites. b) shows the grid pattern in good shape fidelity with a 15 mm×15 mm×2 mm cubic structure that can be 3D printed with pure starch hydrogel. The scale bar is ˜5 mm. c) shows an anatomical ear printed using a nanocomposite starch-based hydrogel with good shape fidelity. The printing temperature was maintained at 6° C. The demonstration of 3D printing of the nanoparticle/hydrogel composites shows the capability of creating 3D compartments with scaffolding biomaterials with precise control. The coloring in the image is from food dye. The scale bar is ˜ 1 cm. d) shows NIH 3T3 cell spreading on a 3D printed nanocomposite starch hydrogel scaffold over a 7-day culture, indicating the ability to promote cell growth and attachment. (The color scale in the legend indicates the printability of the materials).

FIG. 4 shows how starch is composed of two major components: amylose and amylopectin (left-hand image). Amylose is linear or slightly branched while amylopectin is highly branched. The right-hand image shows the process of starch hydrogel preparation and the structural changes in each phase (granules, gelatinization and retrogradation).

FIG. 5 shows the rheological characterization of starch, the starch-collagen blend, and the nanoparticle/hydrogel composites during the extrusion condition (6° C.) a) Images of printed grid patterns with 15 mm×15 mm×2 mm cubic structure (10 layers) using various combinations of bio-inks. From left to right: starch hydrogel, starch-collagen blend, starch-collagen-5G and starch-collagen-10G. b) Shear rate sweep of cornstarch, the cornstarch-collagen blend, and the nanoparticle/hydrogel composites. c) Frequency sweep indicates G′ (closed symbol) and G″ (open symbol) of starch, the starch-collagen blend, and the nanoparticle/hydrogel composites at varied concentrations. d) Representative recovery performance and thixotropy profiles from tested material groups. (S=starch; C=collagen; G=gelatin nanoparticles). The number in front of each letter indicates the final concentration of each component. Starch concentration: 7.5%, 10%, 12.5% w/v; Collagen: 1.33 mg mL-1; Gelatin nanoparticle: 5G: 3.33E9 and 10G: 6.67E9 particles mL-1) FIG. 6 shows a plot of the shear rate sweep of nanoparticle/hydrogel composites with varied combinations of GNPs and starch.

FIG. 7 shows a) Printability characterization of starch, the starch-collagen blend, and the nanoparticle/hydrogel composites b) Images of printed grid patterns (15×15 mm) using various combinations of bio-inks for printability analysis (N=4). c) Structure integrity of starch, the starch-collagen blend, and the nanoparticle/hydrogel composites (N=6) in PBS over 14-day incubation. (S=starch; C=collagen; G=gelatin nanoparticles). The number in front of each letter indicates the final concentration of each component. Starch concentration: 12.5% w/v; Collagen: 1.33 mg mL⁻¹; Gelatin nanoparticle: 5G: 3.33E9 and 10G: 6.67E9 particles mL-1).

FIG. 8 shows Cryo-SEM images, which present hierarchical porous microstructures from a) pure cornstarch (scale bar: 5 um), and b) and c) hybridized hierarchical pores and fibrous sprouts from starch-collagen blend (scale bar: 5 and 3 um), d) gelatin nanoparticles in HEPES buffer (scale bar: 1 um), as well as the microstructures from nanoparticle/hydrogel composites (e and f scale bar: 5 and 2 um). g) shows the gelatin nanoparticles in and f) illustrates the fibrous collagen in the nanoparticle/hydrogel composites.

FIG. 9 shows cryo-SEM images of starch, starch-collagen blend, and nanocomposite hydrogel at a high magnification.

FIG. 10 shows the distribution of red cell tracker-labeled NIH 3T3 cells after seeding on the top of the starch-collagen scaffolds and the nanocomposite scaffolds for 3 h.

FIG. 11 shows a summary of the cell adhesion response of printed nanoparticle/hydrogel composite scaffolds. Starch-collagen blend hydrogel scaffold served as the control group. Limited cell growth on starch-collagen blend hydrogel scaffold on Day 4 (A and C) and Day 7 (B, D and I), compared with abundant cell attachment and spreading on 3D printed nanocomposite starch bio-ink on Day 4 (E and G) and Day 7 (F, H and J). (K). Evaluation of cell proliferation on both scaffolds over a 7-day culture (n=6). (S=starch; C=collagen; G=gelatin nanoparticles). The number in front of each letter indicates the final concentration of each component. Starch concentration: 12.5% w/v; Collagen: 1.33 mg mL-1; Gelatin nanoparticle: 10G: 6.67E9 particles mL⁻¹).

FIG. 12 shows monochrome images of immunofluorescence staining from FIG. 11.

### DETAILED DESCRIPTION

The subject matter described herein relates to nanocomposite hydrogel compositions for 3D (three-dimensional) printing. The 3D printable nanocomposite starch hydrogels, which are formulated with natural collagen and gelatin nanoparticles, exhibit enhanced biocompatibility for promoting 3D cell growth.

Natural extracellular matrix (ECM) biomaterials, such as collagen, undergo irreversible deformation when extruded after gelation without the ability to retain the material shear thinning. Furthermore, natural ECM materials have low stiffness, which significantly limits the ability to form free-standing structures for maintaining 3D shape fidelity (Osidak et al., 2020). In an effort to increase the diversity of mechanically competent materials for 3D bioprinting, modifications, such as the addition of various rheological modifiers, post-printing, and on-site chemical/photo-crosslinking, have been investigated (S. Zhang et al., 2020; Zhuang et al., 2019). However, such crosslinking steps may impair the biocompatibility of developed 3D bio-ink by introducing exogenous chemical groups. This could result in toxicity and immunogenicity in both in vitro and in vivo applications, or cause a mechanically incompliant network that impedes desired cellular responses. Given the requirement of both desired mechanical properties and biological function, using naturally derived biomaterials without any chemical modification for 3D printing is challenging (Ouyang et al., 2020).

Due to its cost-effectiveness, availability, biocompatibility, and biodegradability under physiological conditions, starch has been identified as an appealing natural biomaterial for tissue regeneration and drug delivery (Chen et al., 2019; Perez et al., 2018). However, as a result of its mechanical instability and lack of cell binding sites, starch has not been widely used in tissue engineering. To improve cell adhesion ability and thermal stability from starch, other polymers, such as PVA, chitosan, and collagen, have been introduced to form starch-based blends (Amal et al., 2015; Shi et al., 2010; Wen et al., 2020). Through physically associated networks, starch-based blends with improved mechanical strength, biocompatibility, and processability have been achieved. Early studies demonstrated the utility of starch-based scaffolds for bone tissue engineering (Mirab et al., 2018; Wu et al., 2017). Recently, investigations have focused on the therapeutic potential of wound healing of starch-based scaffolds (Eskandarinia et al., 2019; Torres et al., 2013). Various types of starch have been tailored into scaffolding preparation methods, such as electrospinning, salt leaching, and inject molding (Mao et al., 2020; Waghmare et al., 2018; Wen et al., 2020). Investigations have also demonstrated the use of starch hydrogel for 3D bioprinting (Chen et al., 2019). However, the studies either utilized starch with a concentration of over 25% to secure the mechanical stiffness, or with limited 3D printing fidelity in a low concentration range (Butler et al., 2020).

The nanocomposite hydrogel compositions described herein offer many advantages over the art. Here, gelatin nanoparticles and collagen are incorporated into starch hydrogel for enhancing 3D cell culture and tissue scaffolding. Although nanoparticles, including nanosilicates, starch or cellulose nanocrystals, gold nanoparticles, carbon nanotube, and graphene oxide have been investigated and incorporated into hydrogel networks to achieve desirable material properties (e.g., electrical conductivity, printability, mechanical stiffness, and stimuli response), using gelatin nanoparticles in starch hydrogel for enhancing 3D cell culture and tissue scaffolding ability has not been investigated (A. Nademezhad et al. ACS Appl. Bio Mater. 2019, 2, 796; S. A. Wilson et al. ACS Appl. Mater. Interfaces 2017, 9, 43449; S. Zhang et al., Biomacromolecules 2020, 21, 2400; S. Piluso et al. Biomacromolecules 2019, 20, 3819; B. Begines, et al., Sci. Rep. 2019, 9, 16097; K. Behera et al. Polymer 2020, 186, 122002; Z. Cheng, L. Xigong, et al. 2020, 18, 97). Incorporating gelatin nanoparticles and collagen into a starch-based hydrogel allows for nanoscale topological features, enabling cells to reside in a highly biocompatible microenvironment for interactive binding and attachment. This is essential in regulating 3D cellular response and tissue growth. High 3D printing fidelity is achieved through dynamic rheological characterization and printability evaluation. The hydrogel compositions described herein exhibit desirable shear-thinning and thixotropic properties. The results demonstrate that the combination of gelatin nanoparticles and collagen can tune the mechanical stiffness as desired for extrusion-based 3D bioprinting. Additional microstructure analysis revealed that the nanocomposite hydrogel encompasses hierarchical porous structures with distinct fibrous collagen distributing throughout the pore surface, which is advantageous for cell attachment and migration. The homogeneous microporous structure with abundant collagen fibers and gelatin nanoparticles interlaced web-like structure not only support efficient mass transport, but also supplies rich attachment sites for promoting 3D cell growth, as evidenced by culturing fibroblast cells with increased proliferation rate.

Current clinical translation of scaffold biomaterials in tissue engineering and regenerative medicine is hindered due to concerns of toxicity and biocompatibility. The chemical and mechanical properties of the biomaterial scaffold must be tested to ensure successful interactions with cells and the surrounding tissue microenvironment, including efficient mass transport and host tissue integration. As described herein, the mechanical strength of the nanocomposite starch bio-ink may be tuned by varying the concentration of each component in the composition, which offers the opportunity to tailor the hydrogel to the specific tissue environment. Furthermore, the developed nanocomposite starch bio-ink is non-modified or non-chemically crosslinked. As such, the starch nanocomposite is biodegradable by converting carbohydrates back into forms that are usable for various biosynthetic and metabolic routes in vivo. Consequently, the printed 3D constructs can be replaced along with cell growth and remodeled to match the complexity of the real tissue microenvironment. Accordingly, the nanocomposite hydrogel compositions described herein have potential for clinical use and commercialization in regenerative medicine and biomimetic tissue engineering.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. Unless otherwise defined, all technical and scientific terms used herein have the same meaning as commonly understood by one of ordinary skill in this field. All publications, patent applications, patents, and other references mentioned herein are incorporated by reference in their entirety. In the event that one or more of the incorporated literatures, patents, and similar materials differs from or contradicts this application, including but not limited to defined terms, term usage, described techniques, or the like, this application controls.

### I. Definitions

As used herein, “and/or” refers to and encompasses any and all possible combinations of one or more of the associated listed items, as well as the lack of combinations when interpreted in the alternative (“or”).

As used herein, the term “about,” when referring to a measurable value such as an amount of a compound or agent of the current subject matter, dose, time, temperature, and the like, is meant to encompass variations of ±20%, ±10%, ±5%, ±1%, ±0.5%, or even ±0.10% of the specified amount.

The terms “approximately,” “essentially,” and “substantially” as used herein represent an amount close to the stated amount that still performs a desired function or achieves a desired result. For example, in some embodiments, as the context may dictate, the terms “approximately”, and “substantially” may refer to an amount that is within less than or equal to 10% of the stated amount. The term “generally” as used herein represents a value, amount, or characteristic that predominantly includes or tends toward a particular value, amount, or characteristic.

As used herein, conditional language used herein, such as, among others, “can,” “could,” “might,” “may,” “e.g.,” and the like, unless specifically stated otherwise or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

As used herein, “ECM” refers to extracellular matrix.

As used herein, “GNPs” refers to gelatin nanoparticles.

As used herein, a “culture” refers to the cultivation or growth of cells, for example, tissue cells, in or on a nutrient medium. As is well known to those of skill in the art of cell or tissue culture, a cell culture is generally begun by removing cells or tissue from a human or other animal, dissociating the cells by treating them with an enzyme, and spreading a suspension of the resulting cells out on a flat surface, such as the bottom of a Petri dish. There the cells generally form a thin layer of cells called a “monolayer” by producing glycoprotein-like material that causes the cells to adhere to the plastic or glass of the Petri dish. A layer of culture medium, containing nutrients suitable for cell growth, is then placed on top of the monolayer, and the culture is incubated to promote the growth of the cells.

A “disease” is a state of health of an animal wherein the animal cannot maintain homeostasis, and wherein if the disease is not ameliorated then the animal's health continues to deteriorate.

A disease is “alleviated” if the severity of a symptom of the disease, the frequency with which such a symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

The terms “patient,” “subject,” “individual,” and the like are used interchangeably herein, and refer to any animal, or cells thereof whether in vitro or in situ, amenable to the methods described herein. In certain nonlimiting embodiments, the patient, subject or individual is a mammal, and in other embodiments, the mammal is a human.

“Proliferation” is used herein to refer to the reproduction or multiplication of similar forms, especially of cells. That is, proliferation encompasses production of a greater number of cells, and may be measured by, among other things, simply counting the numbers of cells, measuring incorporation of 3H-thymidine into the cell, and the like.

As used herein, “scaffold” refers to a structure, comprising a biocompatible material that provides a surface suitable for adherence and proliferation of cells. A scaffold may further provide mechanical stability and support. A scaffold may be in a particular shape or form so as to influence or delimit a three-dimensional shape or form assumed by a population of proliferating cells. In embodiments, the scaffold is a hydrogel.

As used herein, “tissue engineering” refers to the process of generating a tissue ex vivo for use in tissue replacement or reconstruction. Tissue engineering is an example of “regenerative medicine,” which encompasses approaches to the repair or replacement of tissues and organs by incorporation of cells, gene, or other biological building blocks, along with bioengineered materials and technologies.

Unless otherwise specified, the terms “composition” and “formulation” as used herein are intended to encompass a product comprising the specified ingredient(s) (and in the specified amount(s), if indicated), as well as any product which results, directly or indirectly, from combination of the specified ingredient(s) in the specified amount(s). Additionally, the terms “composition” and “formulation” refer to a mixture of compounds or particles.

Additional definitions are provided below.

### II. Nanocomposite Hydrogel Compositions

In one aspect, the subject matter described herein is directed to a nanocomposite hydrogel composition for three-dimensional printing, comprising: collagen, starch, and gelatin nanoparticles. The hydrogel presents a tissue engineering scaffold useful in useful in wound healing and tissue regeneration.

In certain embodiments of the nanocomposite hydrogel composition, the collagen is present in the composition at a concentration of about 0.1 mg/mL to 15 mg/mL. In certain embodiments, the collagen is present in the composition at a concentration of about 0.5 mg/mL to 12 mg/mL, 0.7 mg/mL to 14 mg/mL, 0.2 mg/mL to 7 mg/mL, 1 mg/mL to 5 mg/mL, 1.1 mg/mL to 1.7 mg/mL, 1.2 mg/mL to 1.5 mg/mL, 1 mg/mL to 3 mg/mL, 0.8 mg/mL to 4 mg/mL, 1.2 mg/mL to 8 mg/mL, 1 mg/mL to 2 mg/mL, or 1 mg/mL to 10 mg/mL. In certain embodiments, the collagen is present in the composition at a concentration of about 1.00 mg/mL, 1.10 mg/mL, 1.20 mg/mL, 1.21 mg/mL, 1.22 mg/mL, 1.23 mg/mL, 1.24 mg/mL, 1.25 mg/mL, 1.26 mg/mL, 1.27 mg/mL, 1.28 mg/mL, 1.29 mg/mL, 1.30 mg/mL, 1.31 mg/mL, 1.32 mg/mL, 1.33 mg/mL, 1.34 mg/mL, 1.35 mg/mL, 1.36 mg/mL, 1.37 mg/mL, 1.38 mg/mL, 1.39 mg/mL, 1.40 mg/mL, 1.50 mg/mL, 1.60 mg/mL, 1.70 mg/mL, 1.80 mg/mL, 1.90 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, or 15 mg/mL.

In certain embodiments of the nanocomposite hydrogel composition, the starch is present in the composition at a concentration of about 2% w/v to 25% w/v. In certain embodiments, the starch is present in the composition at a concentration of about 2% w/v to 15% w/v, 3% w/v to 7% w/v, 3% w/v to 13% w/v, 4% w/v to 20% w/v, 5% w/v to 15% w/v, 7% w/v to 13% w/v, 7% w/v to 15% w/v, 10% w/v to 22% w/v, 9% w/v to 18% w/v, 12% w/v to 23% w/v, 7% w/v to 18% w/v, 10% w/v to 15% w/v, or 3% w/v to 24% w/v. In certain embodiments, the starch is present in the composition at a concentration of about 2.0% w/v, 3.0% w/v, 4.0% w/v, 5.0% w/v, 6.0% w/v, 7.0% w/v, 7.1% w/v, 7.2% w/v, 7.3% w/v, 7.4% w/v, 7.5% w/v, 7.6% w/v, 7.7% w/v, 7.8% w/v, 7.9% w/v, 8.0% w/v, 8.1% w/v, 8.2% w/v, 8.3% w/v, 8.4% w/v, 8.5% w/v, 8.6% w/v, 8.7% w/v, 8.8% w/v, 8.9% w/v, 9.0% w/v, 9.1% w/v, 9.2% w/v, 9.3% w/v, 9.4% w/v, 9.5% w/v, 9.6% w/v, 9.7% w/v, 9.8% w/v, 9.9% w/v, 10% w/v, 10.1% w/v, 10.2% w/v, 10.3% w/v, 10.4% w/v, 10.5% w/v, 10.6% w/v, 10.7% w/v, 10.8% w/v, 10.9% w/v, 11% w/v, 11.1% w/v, 11.2% w/v, 11.3% w/v, 11.4% w/v, 11.5% w/v, 11.6% w/v, 11.7% w/v, 11.8% w/v, 11.9% w/v, 12% w/v, 12.1% w/v, 12.2% w/v, 12.3% w/v, 12.4% w/v, 12.5% w/v, 12.6% w/v, 12.7% w/v, 12.8% w/v, 12.9% w/v, 13.0% w/v, 13.1% w/v, 13.2% w/v, 13.3% w/v, 13.4% w/v, 13.5% w/v, 13.6% w/v, 13.7% w/v, 13.8% w/v, 13.9% w/v, 14.0% w/v, 15.0% w/v, 16.0% w/v, 17.0% w/v, 18.0% w/v, 19.0% w/v, 20.0% w/v, 21.0% w/v, 22.0% w/v, 23.0% w/v, 24.0% w/v, or 25.0%. In certain embodiments, the starch is present in the composition at a concentration of about 7.5% w/v, 10% w/v, or 1²0.5% w/v. In certain embodiments, the starch is present in the composition at a concentration of at least or about 10% w/v.

In certain embodiments of the nanocomposite hydrogel composition, the gelatin nanoparticles are present in the composition at a concentration of about 1*10⁷ particles/mL to 10*10¹³ particles/mL. In certain embodiments, the gelatin nanoparticles are present in the composition at a concentration of about 2*10⁸ particles/mL to 5*10¹⁰ particles/mL, 2*10⁹ particles/mL to 1*10¹² particles/mL, 5*10⁸ particles/mL to 5*109 particles/mL, 6*10⁷ particles/mL to 8*10¹² particles/mL, or 3*10⁹ to 12*109 particles/mL. In certain embodiments, the gelatin nanoparticles are present in the composition at a concentration of about 1*109 particles/mL, 2*109 particles/mL, 3*109 particles/mL, 4*109 particles/mL, 5*109 particles/mL, 6*109 particles/mL, 7*109 particles/mL, 8*109 particles/mL, 9*109 particles/mL, 10*109 particles/mL, 11*109 particles/mL, 12*109 particles/mL, 13*109 particles/mL, 14*109 particles/mL, or 15*109 particles/mL.

In certain embodiments of the nanocomposite hydrogel composition, the composition comprises starch at a concentration of about 7.5% w/v, collagen at a concentration of about 1.33 mg/mL, and gelatin nanoparticles at a concentration of about 5*109 particles/mL. In certain embodiments of the nanocomposite hydrogel composition, the composition comprises starch at a concentration of about 7.5% w/v, collagen at a concentration of about 1.33 mg/mL, and gelatin nanoparticles at a concentration of about 10*10⁹ particles/mL. In certain embodiments of the nanocomposite hydrogel composition, the composition comprises starch at a concentration of about 10% w/v, collagen at a concentration of about 1.33 mg/mL, and gelatin nanoparticles at a concentration of about 5*109 particles/mL. In certain embodiments of the nanocomposite hydrogel composition, the composition comprises starch at a concentration of about 10% w/v, collagen at a concentration of about 1.33 mg/mL, and gelatin nanoparticles at a concentration of about 10*109 particles/mL. In certain embodiments of the nanocomposite hydrogel composition, the composition comprises starch at a concentration of about 12.5% w/v, collagen at a concentration of about 1.33 mg/mL, and gelatin nanoparticles at a concentration of about 5*109 particles/mL. In certain embodiments of the nanocomposite hydrogel composition, the composition comprises starch at a concentration of about 12.5% w/v, collagen at a concentration of about 1.33 mg/mL, and gelatin nanoparticles at a concentration of about 10*109 particles/mL.

In certain embodiments of the nanocomposite hydrogel composition, the gelatin nanoparticles have a peak particle size of about 1 nm to about 500 nm as determined by NTA analysis. In certain embodiments of the nanocomposite hydrogel composition, the gelatin nanoparticles have a peak particle size of about 5 nm to about 400 nm, 10 nm to about 300 nm, 20 nm to about 10 nm, 30 nm to about 350 nm, 10 nm to about 100 nm, 100 nm to about 450 nm, 30 nm to about 150 nm, 50 nm to about 400 nm, 10 nm to about 200 nm, 35 nm to about 75 nm, 100 nm to about 150 nm, 100 nm to about 200 nm, 100 nm to about 400 nm, 30 nm to about 400 nm, 15 nm to about 60 nm, 20 nm to about 80 nm, 25 nm to about 125 nm, 35 nm to about 165 nm, or 30 nm to about 130 nm. In certain embodiments of the nanocomposite hydrogel composition, the gelatin nanoparticles have a peak particle size of about 30 nm, 31 nm, 32 nm, 33 nm, 34 nm, 35 nm, 36 nm, 36 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 69 nm, or 60 nm as determined by NTA (nanoparticle tracking analysis). In certain embodiments of the nanocomposite hydrogel composition, the gelatin nanoparticles have a peak particle size of about 101 nm, 102 nm, 103 nm, 104 nm, 105 nm, 106 nm, 107 nm, 108 nm, 109 nm, or 110 nm as determined by Dynamic Light Scattering.

Hydrogels can generally absorb a great deal of fluid and, at equilibrium, typically are composed of 60-90% fluid and only 10-30% polymer. Hydrogels are particularly useful due to the inherent biocompatibility of the cross-linked polymeric network (Hill-West, et al., 1994, Proc. Natl. Acad. Sci. USA 91:5967-5971). Hydrogel biocompatibility may be attributed to hydrophilicity and ability to imbibe large amounts of biological fluids (Brannon-Peppas. Preparation and Characterization of Cross-linked Hydrophilic Networks in Absorbent Polymer Technology, Brannon-Peppas and Harland, Eds. 1990, Elsevier: Amsterdam, pp 45-66; Peppas and Mikos. Preparation Methods and Structure of Hydrogels in Hydrogels in Medicine and Pharmacy, Peppas, Ed. 1986, CRC Press: Boca Raton, Fla., pp 1-27). Methods for preparing the nanocomposite hydrogel compositions herein are described in the examples.

Non-limiting types of collagen used in the nanocomposite hydrogel composition include collagen types I, II, III, IV, V, VI, VII, VIII, IX, X, XI, XII, XIII, XIV, XV, XVI, XVII, XVIII, and XIX. In certain embodiments, collagen type I is used.

In certain embodiments of the nanocomposite hydrogel composition, the composition is characterized by at least one of the following properties: printability, shear-thinning, shape fidelity, and thixotropy. In certain embodiments of the nanocomposite hydrogel composition, the composition is characterized by at least two of the following properties: printability, shear-thinning, shape fidelity, and thixotropy. In certain embodiments of the nanocomposite hydrogel composition, the composition is characterized by at least three of the following properties: printability, shear-thinning, shape fidelity, and thixotropy. In certain embodiments of the nanocomposite hydrogel composition, the composition is characterized by all four of the following properties: printability, shear-thinning, shape fidelity, and thixotropy.

As used herein, printability (Pr), which demonstrates the gelation of the bio-ink, may be calculated using the following equation: Pr=L² 16A. In certain embodiments, the nanocomposite hydrogel compositions described herein can be characterized by a Pr value of at least 0.800. In certain embodiments, the nanocomposite hydrogel compositions described herein have a Pr value of at least or about 0.800, 0.810, 0.820, 0.830, 0.840, 0.841, 0.842, 0.843, 0.844, 0.845, 0.846, 0.847, 0.848, 0.849, 0.850, 0.851, 0.852, 0.853, 0.854, 0.855, 0.856, 0.857, 0.858, 0.859, 0.860, 0.861, 0.862, 0.863, 0.864, 0.865, 0.866, 0.867, 0.868, 0.869, 0.890, 0.891, 0.892, 0.893, 0.894, 0.895, 0.896, 0.897, 0.898, 0.899, 0.900, 0.901, 0.902, 0.903, 0.904, 0.905, 0.906, 0.907, 0.908, 0.909. 0.910, 0.911, 0.912, 0.913, 0.914, 0.915, 0.916, 0.917, 0.918, 0.919, 0.920, 0.921, 0.922, 0.923, 0.924, 0.925, 0.926, 0.927, 0.928, 0.929, 0.930, 0.931, 0.932, 0.933, 0.934, 0.935, 0.936, 0.937, 0.938, 0.939, 0.940, 0.941, 0.942, 0.945, 0.946, 0.947, 0.948, 0.949, or 0.945.

As used herein, shear thinning refers to the non-Newtonian behavior of fluids whose viscosity decreases under shear strain.

As used herein, shape fidelity refers to the ability of a material to maintain its shape after printing. Shape fidelity can be evaluated visually through imaging techniques, such as magnetic resonance Imaging, X-rays, or computed tomography.

As used herein, thixotropy refers to the property of becoming less viscous when subjected to an applied stress, shown for example by some gels which become temporarily fluid when shaken or stirred.

In embodiments, the nanocomposite hydrogel compositions described herein exhibit mechanical strength. As used herein, mechanical strength refers to a material's ability to effectively support cell growth, including efficient mass transport and host tissue integration.

In certain embodiments of the nanocomposite hydrogel composition, the composition is characterized by an interconnected, dense porous structure. In certain embodiments, the composition is characterized by a homogeneous microporous structure.

### III. Three-Dimensional Printed Objects

In embodiments, the subject matter disclosed herein is directed to a three-dimensional object printed from the nanocomposite hydrogel compositions described herein. In certain embodiments, the three-dimensional object is structurally integrous and stable.

As used herein, a material that is structurally integrous refers to the material's ability to maintain the desired shape during and after printing. In certain embodiments, a structurally integrous material or object is characterized by constant width and smooth edges in the shape of the extrusion path without bulging, thinning, or breaking.

As used herein, a material that is stable is able to support a long term cell culture. In certain embodiments, the stability of a material can be measured by its immersion in PBS, wherein the material does not deform or collapse over a period of time. As shown herein, the objects printed from the nanocomposite hydrogel compositions are stable for at least one hour, two hours, five hours, 1 day, 2 days, 3 days, 4 days, 5 days, 6 days, 7 days, 8 days, 9 days, 10 days, 11 days, 12 days, 13 days, and up to 14 days.

In certain embodiments of the three-dimensional object printed from the nanocomposite hydrogel composition described herein, the three-dimensional object is biocompatible. As used here, “biocompatible” refers to any material, which, when implanted in a mammal, does not provoke an adverse response in the mammal. A biocompatible material, when introduced into an individual, is not toxic or injurious to that individual, nor does it induce immunological rejection of the material in the mammal. As described herein, the present nanocomposite hydrogel compositions are not chemically modified or chemically crosslinked. As such, risk of toxicity or immunogenicity in both in vitro and in vivo applications is low.

In certain embodiments of the three-dimensional object printed from the nanocomposite hydrogel composition described herein, the three-dimensional object is biodegradable. In general, a biodegradable substance is one that can be broken down under physiological conditions. In certain embodiments, a material that is biodegradable is capable of converting carbohydrates back into forms that are usable for various biosynthetic and metabolic routes.

In certain embodiments of the three-dimensional object printed from the nanocomposite hydrogel composition described herein, the three-dimensional object is not chemically modified. For example, in certain embodiments, the three-dimensional object is not chemically cross-linked.

In certain embodiments of the three-dimensional object printed from the nanocomposite hydrogel composition described herein, the object is for use in tissue engineering, wherein the object promotes cell attachment and growth.

In certain embodiments of the three-dimensional object printed from the nanocomposite hydrogel composition described herein, the object is printed by extrusion printing. With extrusion deposition, small beads of material are extruded from a nozzle to be fused to material that has already been laid down. By extruding successive layers of beads of material through a nozzle under the control of one or more controller driven motors, it is possible to form articles with highly complex shapes that have heretofore not been possible, or prohibitively expensive to manufacture. Methods for printing three-dimensional objects are described in the examples.

### IV. Methods of Treatment

In certain embodiments, the subject matter described herein is directed to a method of treating a wound and/or regenerating tissue in a subject in need thereof, comprising administering to a treatment site in said subject the nanocomposite hydrogel compositions described herein.

The term “wound” as used herein refers to all types of tissue injuries, including those inflicted by surgery and trauma, including burns, as well as injuries from chronic medical conditions, such as atherosclerosis, vascular disease, or diabetes. The compositions described herein are useful for treatment of all types of wounds, including wounds to internal and external tissues.

As used herein, “treating a wound” refers to healing or ameliorating a wound in a patient, comprising administering a composition comprising the nanocomposite hydrogel described herein.

As used herein, “regenerating tissue” refers to the process of regenerating or redeveloping tissue, such as, but not limited to skin, bone, cartilage, myocardium, and/or fat.

In certain embodiments of the above method, the nanocomposite hydrogel is printed at the treatment site to produce a three-dimensional object. In certain embodiments of the above method, the treatment site is on an external surface of the subject. For example, on the skin of a patient. In certain embodiments of the above method, the treatment site is at an internal location within the subject. For example, a surgical site inside a subject.

In certain embodiments, the subject matter described herein includes pharmaceutical compositions comprising the nanocomposite hydrogel compositions. Formulations may be employed in admixtures with conventional excipients, i.e., pharmaceutically acceptable organic or inorganic carrier substances suitable for administration to the wound or treatment site. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., other analgesic agents.

In certain embodiments, the nanocomposite hydrogel compositions may be modified with functional groups for covalently attaching a variety of compounds such as therapeutic agents. Therapeutic agents which may be linked to the matrix include, but are not limited to, analgesics, anesthetics, antifungals, antibiotics, anti-inflammatories, anthelmintics, antidotes, antiemetics, antihistamines, antihypertensives, antimalarials, antimicrobials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, sedatives, sulfonamides, stimulants, sympathomimetics, tranquilizers, urinary anti-infectives, vasoconstrictors, vasodilators, vitamins, xanthine derivatives, and the like. The therapeutic agent may also be other small organic molecules, naturally isolated entities or their analogs, organometallic agents, chelated metals or metal salts, peptide-based drugs, or peptidic or non-peptidic receptor targeting or binding agents. It is contemplated that linkage of the therapeutic agent to the matrix may be via a protease sensitive linker or other biodegradable linkage. Molecules which may be incorporated into the nanocomposite hydrogel composition matrix include, but are not limited to, vitamins and other nutritional supplements; fibronectin; peptides and proteins; carbohydrates (both simple and/or complex); proteoglycans; antigens; oligonucleotides (sense and/or antisense DNA and/or RNA); antibodies (for example, to infectious agents, tumors, drugs or hormones); and gene therapy reagents. In other embodiments, the therapeutic agents could be small molecule drugs, antibody/protein or biological drugs, encapsulate and/or attach extracellular vesicles and exosomes, hormones, growth or stimulating factors, and stem cells or other cells.

### V. Articles of Manufacture

In another aspect, described herein are articles of manufacture, for example, a “kit” containing materials useful for treating wounds and/or regenerating tissue in a subject in need thereof. In certain embodiments, the kit comprises a vial containing a nanocomposite hydrogel composition for three-dimensional printing, comprising: collagen, starch, and gelatin nanoparticles. The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister packs, etc. The container may be formed from a variety of materials such as glass or plastic. The label or package insert indicates that the composition is useful for treating wounds and/or regenerating tissue. The article of manufacture may further include other materials desirable from a commercial and user standpoint, including diluents, filters, needles, and syringes.

The kit may further comprise directions for the administration of the composition. For example, the kit many contain directions describing the extrusion printing process of the nanocomposite hydrogel composition for generating a three-dimensional object for wound healing and/or regenerating tissue.

In certain other embodiments the individual components of the kit may comprise a container for containing the separate compositions such as a divided bottle or a divided foil packet, however, the separate compositions may also be contained within a single, undivided container. Typically, the kit comprises directions for the use of the separate components.

### VI. Methods of Preparing the Nanocomposite Hydrogel Compositions

In certain embodiments, the subject matter described herein is directed to methods for preparing the nanocomposite hydrogel compositions described herein, comprising:
contacting starch with a mixture comprising gelatin nanoparticles and water to form a first mixture;
cooling the first mixture; and,
contacting the first mixture with collagen,
   wherein the nanocomposite hydrogel composition is prepared.

In embodiments for preparing the nanocomposite hydrogel compositions, the method does not comprise chemical crosslinking. As used herein, chemical crosslinking also encompasses photo-crosslinking.

In certain embodiments for preparing the nanocomposite hydrogel compositions, contacting starch with the mixture comprising gelatin nanoparticles and water is at a temperature of about 0° C. to 100° C., 5° C. to 50° C., 10° C. to 75° C., 15° C. to 60° C., 10° C. to 90° C., or 25° C. to 65° C. In certain embodiments for preparing the nanocomposite hydrogel compositions, contacting starch with the mixture comprising gelatin nanoparticles and water is at a temperature of about 40° C., 41° C., 42° C., 43° C., 44° C., 45° C., 46° C., 47° C., 48° C., 49° C., 50° C., 51° C., 52° C., 53° C., 54° C., or 55° C. In certain embodiments for preparing the nanocomposite hydrogel compositions, the contacting starch with the mixture comprising gelatin nanoparticles and water proceeds for about 15 minutes to about 60 minutes or 5 minutes to about 40 minutes. In certain embodiments for preparing the nanocomposite hydrogel compositions, the contacting starch with the mixture comprising gelatin nanoparticles and water proceeds for about 15, 16, 17, 18, 19, 20, 21, 22, 23, 24, 25, 26, 27, 28, 29, or 30 minutes.

In certain embodiments for preparing the nanocomposite hydrogel compositions, the cooling is conducted at a temperature of about −3° C. to 10° C. In certain embodiments for preparing the nanocomposite hydrogel compositions, the cooling is conducted at a temperature of about 0° C., 1° C., 2° C., 3° C., 4° C., 5° C., 6° C., 7° C., or 8° C.

In certain embodiments for preparing the nanocomposite hydrogel compositions, the cooling proceeds for about 1 hour to about 4 hours, 1 hour to about 3 hours, 1 hour to about 2 hours, 1.5 hours to 3.5 hours, or from about 40 minutes to about 80 minutes. In certain embodiments for preparing the nanocomposite hydrogel compositions, the cooling proceeds for about 55 minutes, 56 minutes, 57 minutes, 58 minutes, 59 minutes, 60 minutes, 61 minutes, 62 minutes, 63 minutes, 64 minutes, or 65 minutes.

In certain embodiments for preparing the nanocomposite hydrogel compositions, the contacting the first mixture with collagen proceeds for about 30 minutes to about 6 hours. In certain embodiments for preparing the nanocomposite hydrogel compositions, the contacting the first mixture with collagen proceeds for about 30 minutes, 45 minutes, 1 hour, 1.5 hours, 2 hours, 2.5 hours, 3 hours, 3.5 hours, 4 hours, 4.5 hours, 5 hours, 5.5 hours, or 6 hours.

In certain embodiments for preparing the nanocomposite hydrogel compositions, the mixture comprising gelatin nanoparticles and water further comprises a buffer. In certain embodiments for preparing the nanocomposite hydrogel compositions, the nanocomposite hydrogel composition is cooled at a temperature of about −3° C. to 10° C. for a duration of about 40 minutes to about 80 minutes prior to printing.

In certain embodiments for preparing the nanocomposite hydrogel compositions, the starch is contacted with a buffer prior to said contacting with said mixture comprising gelatin nanoparticles and water.

In certain embodiments for preparing the nanocomposite hydrogel compositions, the collagen is contacted with a buffer prior to said contacting with said first mixture.

In certain embodiments for preparing the nanocomposite hydrogel compositions, prior to contacting starch with a mixture comprising gelatin nanoparticles and water to form a first mixture, the gelatin nanoparticles are prepared by contacting gelatin with water at a temperature of about 0° C. to about 100° C. for about 30 minutes to 6 hours to form a pre-mixture; contacting the premixture with a solvent, such as acetone, allowing gelatin to precipitate from the premixture to form precipitated gelatin; contacting the precipitated gelatin with water at a temperature of about 0° C. to about 100° C. to form a second premixture; adjusting the pH of the second premixture to about 2.7-3.0 with acid; contacting the second premixture with a solvent, such as acetone, and glutaraldehyde; and allowing the second premixture to age for about 2 hours to 15 hours; and then contacting the second premixture with glycine, to prepare the gelatin nanoparticles.

The subject matter described herein is directed to the following embodiments:
1. A nanocomposite hydrogel composition for three-dimensional printing, comprising: collagen, starch, and gelatin nanoparticles.
2. The nanocomposite hydrogel composition of embodiment 1, wherein said collagen is present in said composition at a concentration of about 0.1 to 15 mg/mL.
3. The nanocomposite hydrogel composition of embodiment 1 or 2, wherein said collagen is present in said composition at a concentration of about 1 to 2 mg/mL.
4. The nanocomposite hydrogel composition of any one of embodiments 1-3, wherein said collagen is present in said composition at a concentration of about 1.33 mg/mL.
5. The nanocomposite hydrogel composition of any one of embodiments 1-4, wherein said starch is present in said composition at a concentration of about 2% to 25% w/v.
6. The nanocomposite hydrogel composition of any one of embodiments 1-5, wherein said starch is present in said composition at a concentration of about 7% w/v to 15% w/v.
7. The nanocomposite hydrogel composition of any one of embodiments 1-6, wherein said starch is present in said composition at a concentration of about 12.5% w/v.
8. The nanocomposite hydrogel composition of any one of embodiments 1-7, wherein said gelatin nanoparticles are present in said composition at a concentration of about 1*10⁷ particles/mL to 10*10¹³ particles/mL.
9. The nanocomposite hydrogel composition of any one of embodiments 1-8, wherein said gelatin nanoparticles are present in said composition at a concentration of about 3*109 particles/mL to 12*10⁹ particles/mL.
10. The nanocomposite hydrogel composition of any one of embodiments 1-9, wherein said composition comprises starch at a concentration of about 7.5%, 10%, or 12.5% w/v, collagen at a concentration of about 1.33 mg/mL, and gelatin nanoparticles at a concentration of about 5*10⁹ or 10*10⁹ particles/mL.
11. The nanocomposite hydrogel composition of any one of embodiments 1-10, wherein said composition comprises starch at a concentration of about 12.5% w/v, collagen at a concentration of about 1.33 mg/mL, and gelatin nanoparticles at a concentration of about 10*109 particles/mL.
12. The nanocomposite hydrogel composition of any one of embodiments 1-11, wherein said gelatin nanoparticles have a peak particle size of about 1 nm to about 500 nm as determined by nanoparticle tracking analysis (NTA).
13. The nanocomposite hydrogel composition of any one of embodiments 1-12, wherein said gelatin nanoparticles have a peak particle size of about 40 nm as determined by NTA.
14. The nanocomposite hydrogel composition of any one of embodiments 1-13, wherein said composition is biodegradable.
15. The nanocomposite hydrogel composition of any one of embodiments 1-14, wherein said composition is biocompatible.
16. The nanocomposite hydrogel composition of any one of embodiments 1-15, wherein said composition exhibits at least one of the following properties: printability, shear-thinning, shape fidelity, mechanical strength, and thixotropy.
17. The nanocomposite hydrogel composition of any one of embodiments 1-16, wherein said composition is characterized by an interconnected, dense porous structure.
18. A three-dimensional object printed from the nanocomposite hydrogel composition of any one of embodiments 1-17.
19. The three-dimensional object of embodiment 18, wherein said object is biocompatible.
20. The three-dimensional object of embodiment 18 or 19, wherein said object is biodegradable.
21. The three-dimensional object of any one of embodiments 18-20, wherein said object is not chemically cross-linked.
22. The three-dimensional object of any one of embodiments 18-21, wherein said object is structurally integrous and stable.
23. The three-dimensional object of any one of embodiments 18-22, or said nanocomposite hydrogel composition of any one of embodiments 1-16, for use in tissue engineering, wherein said object or composition promotes cell attachment and growth.
24. The three-dimensional object of any one of embodiments 18-23, wherein said object is printed by extrusion printing.
25. A method of treating a wound and/or regenerating tissue in a subject in need thereof, comprising administering to a treatment site in said subject the nanocomposite hydrogel composition of any one of embodiments 1-16.
26. The method of embodiment 25, wherein the nanocomposite hydrogel composition is printed at the treatment site to produce a three-dimensional object.
27. The method of embodiment 25 or 26, wherein the treatment site is on an external surface of said subject.
28. The method of embodiment 25 or 26, wherein the treatment site is at an internal location within said subject.
29. A method of preparing the nanocomposite hydrogel composition of any one of embodiments 1-16, comprising:
contacting starch with a mixture comprising gelatin nanoparticles and water to form a first mixture;
cooling said first mixture; and,
contacting said first mixture with collagen,
   wherein said nanocomposite hydrogel composition is prepared; and,
   wherein said method does not comprise chemical cross-linking.
   30. The method of embodiment 29, wherein said contacting starch with said mixture comprising gelatin nanoparticles and water is at a temperature of about 0° C. to 100° C.
   31. The method of embodiment 29 or 30, wherein said contacting starch with said mixture comprising gelatin nanoparticles and water proceeds for about 15 to about 60 minutes.
   32. The method of any one of embodiments 29-31, wherein said cooling is at a temperature of about 4° C.
   33. The method of any one of embodiments 29-32, wherein said cooling proceeds for about 1 hour to about 4 hours.
   34. The method of any one of embodiments 29-33, wherein said contacting said first mixture with collagen proceeds for about 30 minutes to about 6 hours.

The following examples are offered by way of illustration and not by way of limitation.

### Examples

### Methods and Materials

### Materials

Commercial pregelatinized native common corn starch (Cargill Gel-Instant 12030) was gratefully supplied by Cargill, USA. Ham's F-12K medium, Dulbecco's Phosphate Buffered Saline, heat inactivated fetal bovine serum, penicillin/streptomycin, Cell Tracker Red CMTPX, LIVE/DEAD™ Viability/Cytotoxicity Kit, PrestoBlue™ Cell Viability Reagent, DAPI and Alexa Fluor™ 488 Phalloidin for cell culture and characterization were purchased from Thermo Fisher Scientific. Collagen Type I from rat tail was purchased from Advanced BioMatrix, Carlsbad, CA (Catalog #5279). Type A gelatin from porcine skin (300 g Bloom), glycine, HEPES and 25% glutaraldehyde solution were obtained from Sigma-Aldrich, USA. Other chemicals including acetone, sodium hydroxide and hydrochloric acid (1N HCL solution) were purchased from Fisher Scientific, USA.

### Preparation of Gelatin Nanoparticles (GNPs)

Gelatin nanoparticles were prepared by a two-step coagulation process based on a previously reported study (S. Ruan et al., Biomaterials 2015, 60, 100; C. J. Coester, et al., J. Microencapsulation 2000, 17, 187.). Briefly, 0.625 g gelatin was dissolved in 12.5 mL DI water at 40° C. under constant stirring for Th. 12.5 mL acetone was introduced to the solution at a syringe pump-driven constant speed of 0.1 mL/s under vigorous stirring (300 rpm). Thereafter, the solution was allowed to sit for 15 min for the high molecular weight gelatin to precipitate, and the supernatant that contained low molecular weight gelatin was decanted. 7 mL of DI water was subsequently added to redissolve the precipitated gelatin at 40° C. Upon complete dissolution, the pH of the solution was brought down to a range of 2.7-3.0 using hydrochloric acid. 16.5 mL acetone was further added to the solution at a rate of 1 mL/min under constant stirring (600 rpm). 1 mL acetone containing 60 μL 25% glutaraldehyde solution was gradually fed to the solution at a rate of 0.05 mL/min to crosslink the gelatin nanoparticles and the solution was stirred overnight. The reaction was deactivated by adding 200 μL of 1M Glycine solution. The acetone was then removed, and the solution was filtered through a 0.22 μm syringe filter. The obtained solution was stored at 4° C. for future use.

### Characterization of Gelatin Nanoparticles (GNPs)

### Nanoparticle Tracking Analysis

Gelatin nanoparticle's size distribution and concentration were determined by Nanoparticle Tracking Analysis (NTA) using a NanoSight NS300 system (Malvern Technologies, Malvern, UK) configured with a 488 nm laser. Samples were diluted 50× in dPBS to an acceptable concentration. Samples were analyzed under constant flow conditions (flow rate=50 μL/min) at 25° C. with a camera level of 8. Data were analyzed using NTA 3.4 software with a detection threshold of 5.

### Dynamic Light Scattering

Dynamic light scattering allows for the material characterization of particle size distributions for nanoparticles in various solutions through the analysis of the electrophoretic mobility. A Malvern Zetasizer (Malvern Panalytical Inc. 117 Flanders Road Westborough MA 01581-1042 United States) was used in conducting dynamic light scattering on gelatin nanoparticles in diluted 50-fold deionized water, having an approximate concentration around 109 particles/mL. In preparing the sample for dynamic light scattering (Malvern Zetasizer), the scattering mode and angle were automatically selected as 900 inside scattering mode, with an equilibration time at 1 min and a temperature of 25° C., before measuring five runs per sample. The sample was prepared in pH 7.2 solution, with the wavelength of the incident laser at 633 nm. A cuvette loading cell was used and all results are listed with an average of five runs.

### Scanning Electron Microscopy

Field Emission-Scanning electron microscopy (FE-SEM) was used for visualizing and probing the gelatin nanoparticles' morphology, topology, and size. Samples were first prepared through a serial dilution to a final concentration of 1×10⁹ particles/mL, followed by homogenization by light vortexing at 3000 RPM for 10 seconds, then directly aliquoted onto a 100% acetone-cleaned, Ted Pella aluminum pin stub mount (4595 Mountain Lakes Blvd, Redding, Ca 96003). The solution was then allowed to evaporate completely. Immediately, the samples were sputter coated for 60 seconds with an Au/Pd target using a Denton Desk V Sputter Coater (1259 North Church St. Bldg 3 Mooretown, NJ USA 08057), and loaded into the Hitachi SU5000 Schottky Field-Emission Scanning Electron Microscope (20770 W. Nordhoff Street, Building 4, Chatsworth, CA 91311, United States) at a high negative vacuum pressure of 10-8 torr. An incident electron beam was applied onto the samples at 7 keV and beam current of 16.7 nA. Aperture and stigmata corrections were done before sample images were obtained.

### Cytotoxicity of GNPs

The cytotoxicity of GNPs was determined using PrestoBlue assay. Nanoparticles were suspended into culture medium to achieve final concentrations at 0, 10², 10⁴, 10⁶, 10⁸, 10¹⁰ particles/mL, respectively. NIH-3T3 cells were plated at a density of 5×10⁴ cells/well in 24 well plate and then incubated with nanoparticle-loaded medium for 5 days. PrestoBlue reagent was added to the medium for a final concentration of 10% and incubated for 2 h at 37° C. The absorbance was measured using Cytation 5, BioTek plate reader (Q=570 nm). The intensity was then converted to cell numbers according to the plotted standard curve at each time point (1, 3, and 5d).

### Preparation of Nanoparticle-Supplemented Starch-Collagen Hydrogel

The starch powder was sterilized by UV radiation thrice before use. The GNPs in DI water were loaded to HEPES buffer to achieve 50 mM HEPES buffer (1×PBS and pH=7.4) containing a series of particle concentrations at 5×10⁹ and, 10×10⁹ particles/mL. Starch was stirred into GNPs-containing HEPES buffer at 45° C. with constant stirring at 400 rpm for 20 min using an overhead mixer. After degassing by centrifugating for 30 min at 3000×g, the GNPs-containing starch gels were kept at 4° C. in a fridge for 1 h. Prior to mixing, the collagen was neutralized with HEPES buffer (2×PBS) in a 1:1 ratio. The homogeneous nanoparticle/hydrogel composites were formed by mixing GNPs-loaded starch hydrogel and collagen through a three-way stop cock in a 2:1 ratio. Thus, nanoparticle/hydrogel composites with 6 different combinations (starch final concentrations at 7.5%, 10%, 12.5% w/v); GNP final concentrations at 3.33×10⁹, 6.67×10⁹ particles/mL; collagen: 1.33 mg/mL) were obtained (as shown in Table 1, the labeling was used in the following experiments). The nanoparticle/hydrogel composites were kept in the fridge for 3 h before use. Starch gels at 7.5% w/v, 10% w/v, 12.5% w/v in HEPES buffer and the starch-collagen hydrogel blends were prepared as a parallel control.

TABLE 1
Composition of the samples and control
groups and their corresponding labeling.

| | | Control | Starch- |
|---|---|---|---|
| | Samples | Pure starch | collagen blend |
| Starch | 7.5%, 10%, | 7.5%, 10%, | 7.5%, 10%, |
| | 12.5% | 12.5% | 12.5% |
| Collagen | 1.33 mg/mL | / | 1.33 mg/mL |
| GNP | 5 × 10⁹, 10 × 10⁹ | / | / |
| Sample | 7.5 S-1.33 C-5 G | 7.5 S | 7.5 S-1.33 C |
| Labeling | 7.5 S-1.33 C-10 G | 10 S | 10 S-1.33 C |
| | 10 S-1.33 C-5 G | 12.5 S | 12.5 S-1.33 C |
| | 10 S-1.33 C-10 G | | |
| | 12.5 S-1.33 C-5 G | | |
| | 12.5 S-1.33 C-10 G | | |

### Rheological Evaluation of the Hydrogel/Nanoparticle Composites

Rheological evaluation of the nanocomposite hydrogels was implemented on the MCR 702 MultiDrive rheometer (Anton Paar), using a 25 mm measuring plate. Strain sweeps, frequency sweeps and shear rate sweeps were carried out at 6° C. Strain sweeps in the range of 0.1%-100% at the frequencies of 1 Hz at 6° C. were performed to identify the linear viscoelastic region (LVR) of the hydrogel samples, followed by frequency sweeps from 0.1 to 100 rad s⁻¹ at a constant 10% strain. Shear rate sweeps in a range of 0.1-500 s⁻¹ were tested subsequently. The thixotropic property was investigated by exerting a shear rate at 0.1 s⁻¹ for 60 s (before printing) to the material, followed by increasing the shear rate to 100 s⁻¹ and maintaining it for 10 s (bio-inks being extruded through the needle tip, high shear rate generated), and finally, decreasing the shear rate to 0.1 s⁻¹ for 60 s (recovery). The viscosity over time was recorded.

### 3D Printing and Structural Integrity Evaluation

All 3D bioprinting was performed with the EnvisionTEC 3D Bioplotter®. Material was loaded into a sterile 30 cc cartridge and extruded through a tapered 25G needle tip (ID=260 μm). The temperature of the printing cartridge was maintained at 6° C. during printing. Printing pressure and speed were manually optimized for each combination. Each construct contained 10 layers. 15 mm×15 mm×2 mm scaffolds in a grid pattern were printed directly into 6-well culture plates. Printability, which demonstrates the gelation of the bio-ink, could be calculated using the following equation:

$$P r = {L}^{2} / 16 A \qquad ( 1 )$$

Where L is the perimeter of the pore and A the area of the pore. To obtain the Pr value of each bio-ink, optical images of printed constructs were captured and analyzed in Image-J software. The perimeter and area of the pores were achieved through threshold adjustment (n=4). To demonstrate the printability of the selected bio-ink, a human anatomical ear shape was printed. The structural integrity of the nanoparticle/hydrogel composites in DPBS were examined for a consecutive 14 days. 3D printed rectangular samples (15 mm×15 mm×2 mm) were prepared as well. Samples were observed at various time intervals. The experiment was conducted (N=6) under identical conditions.

### Microstructure Characterization of the Nanoparticle/Hydrogel Composites

Cryo-FE-SEM (CFE-SEM) was also used for visualizing and probing the pore structures, shapes, sizes, and content for a series of hydrogel composites: Starch, Starch-Collagen, Starch-Collagen-GNPs, and gelatin nanoparticles (GNPs). A 5 mm thick hydrogel (in the z-component) was flash-frozen within a tank of liquid nitrogen and immediately passed into the cryo-box attachment on the Hitachi FE-SEM. Fractionation within the cryo-box, by cleavage on the longitudinal plane, immediately proceeded and the sample was allowed to sublimate off the water under high negative pressure at 10-8 torr, analogous to lyophilization. Aperture and stigmata corrections were done before sample images were obtained. ImageJ was used to analyze the microstructures of the samples.

### Cell Culture and Characterization

NIH 3T3 mouse fibroblasts (American Type Culture Collection, Manassas, VA) were cultured in DMEM supplemented with 10% FBS and 1% penicillin/streptomycin in a humidified incubator at 37° C. with 5% CO₂. The cell culture medium was changed every 2-3 days, and the cells were harvested once they reached 80-90% confluence. Cells were labeled with cell tracker Red CMTPX for 30 min prior to seeding for the ease of visualization. For cell seeding, all the printed scaffolds were soaked in complete cell suspension for 3 h before seeding. Cells were then prepared with a density at 5×10⁶ cells/mL, 40 μL cell suspension droplet was carefully placed onto the center of each scaffold surface; thereafter, the scaffold was incubated for 3-4 h in a CO₂ incubator to allow the cell attachment to the scaffolds. 1 mL medium was gently added to the culture well plate after the cells attached. The medium was changed every day.

Similarly, cell metabolic activity was examined with the PrestoBlue assay. Samples (N=6) were incubated with PrestoBlue at a ratio of 9:1 for 2 h at day 1, 4 and 7. To assess cell attachment and morphology, printed constructs were stained for phalloidin (F-actin in green) and 4′,6-diamidino-2-phenylindole (DAPI; nuclei in blue) at day 4 and 7. Samples were fixed with 4% (v/v) paraformaldehyde for 30 min at room temperature, rinsed 3 times with PBS, then permeabilized with 0.2% (v/v) Triton X-100 for 30 min at room temperature. Afterwards, the samples were thoroughly rinsed 3 times. Blocking buffer containing 5% bovine serum albumin (BSA) and 0.1% (v/v) Triton X-100 in PBS was added and incubated for 1 h. The samples were washed thrice after the removal of blocking buffer. Alexa Fluor™ 488 Phalloidin (0.8 U/mL) and DAPI in PBS were added to each sample and incubated for 2 h and 30 min away from the light to stain F-actin and nuclear, respectively.

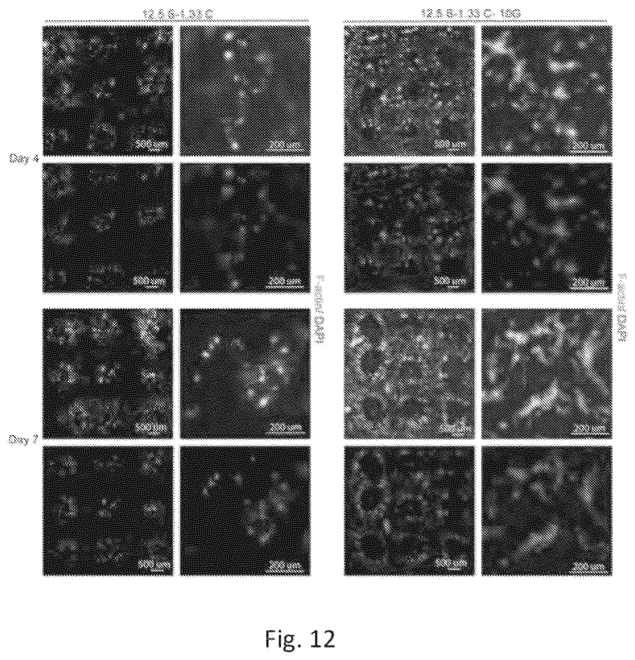

The Cytation 5 Cell Imaging Multi-Mode Reader equipped with an inverted fluorescent microscope (widefield) was used for fluorescence cell imaging. Objectives with 4× and 20× magnifications were used. The fluorescence imaging channel was in the order of FITC followed with DAPI. Images were processed by Gene 5 software with stitching function to achieve FIG. 11. The scaffold material has very light autofluorescence background which is much lower compared to fluorescence signals from labeled cells as shown in FIG. 12. By applying image contrast, cells can be easily differentiated from scaffold materials and their stretching morphology was presented in FIGS. 11 and 12.

### Statistical Analysis

All results are expressed as mean value±standard deviation (SD) with the number of replicates indicated elsewhere. The results were evaluated by one-way ANOVA analysis coupled with a post-hoc Tukey test. Differences are considered statistically significant when p≤0.05 and greatly significant when p≤0.001.

### Example 1: Evaluation of GNPs

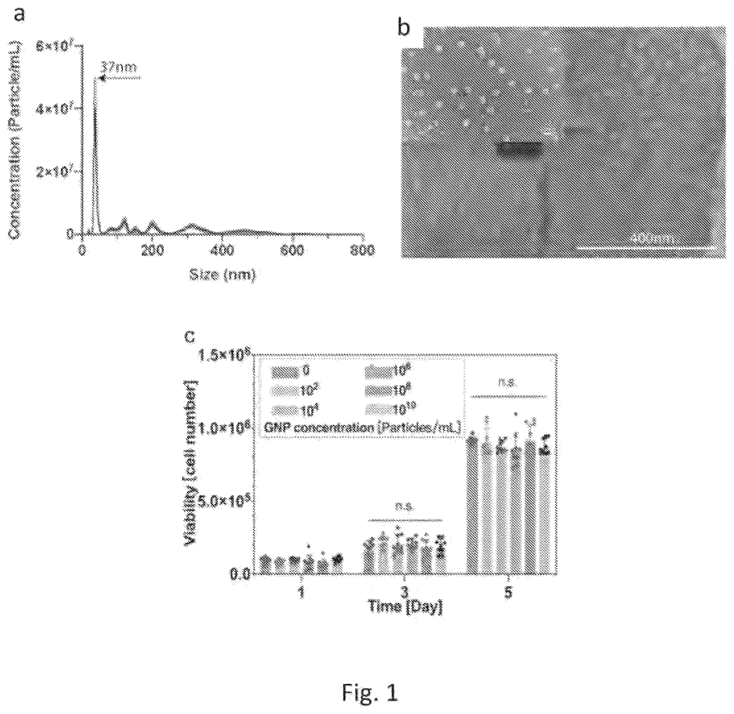

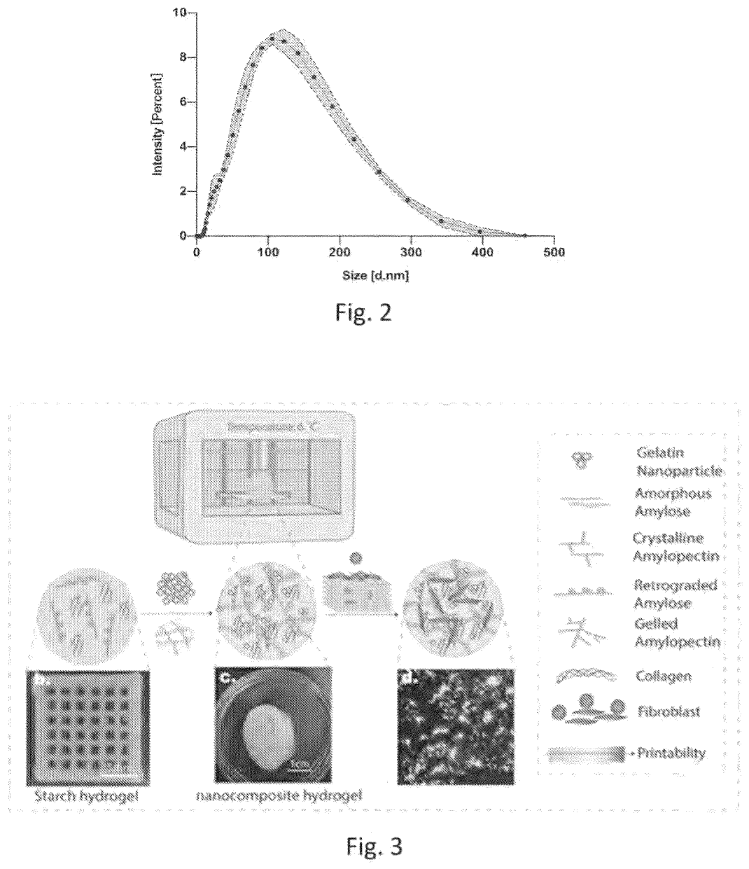

Gelatin nanoparticles were synthesized successfully based on a two-step coagulation process (Ruan et al., 2015). NTA and DLS analysis were employed to determine the size of GNPs, as shown in FIG. 1 and FIG. 2. The results demonstrate a broader distribution and larger particle sizes with DLS (peak particle size ˜106 nm (diameter)) in comparison to NTA (˜40 nm (diameter)). One possible reason is the aggregation or swelling of nanoparticles in DI water. Although both NTA and DLS measure the Brownian movement of the particles, the detection principles differ. NTA tracks the movement of the particles on a particle-by-particle basis under a microscope, while DLS recognizes the scattering intensity fluctuations resulting from the relative Brownian motion of the particles. Therefore, the contribution to the size distribution from the smaller particles could be overshadowed by the higher scattering signals from the larger particle aggregates. As indicated by SEM imaging (FIG. 3), the sizes of gelatin nanoparticle were close to ˜40.

To evaluate the cytotoxicity of the gelatin nanoparticles, gelatin nanoparticles with varying concentrations spanning from 0 to 10¹⁰ particles/mL were incubated with NIH 3T3 cells. As evidenced in inset d in FIG. 3, the cell proliferation rates in the groups with nanoparticles were comparable to the non-GNPs control group, highlighting the negligible toxicity of the gelatin nanoparticles on cells.

### Example 2: Preparation of GNP-Supplemented Starch-Collagen Hydrogels

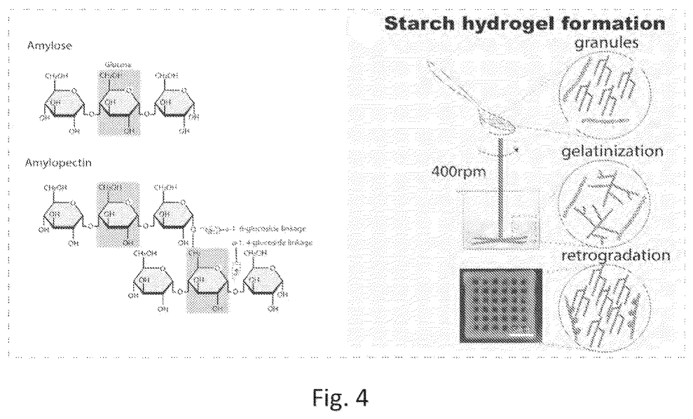

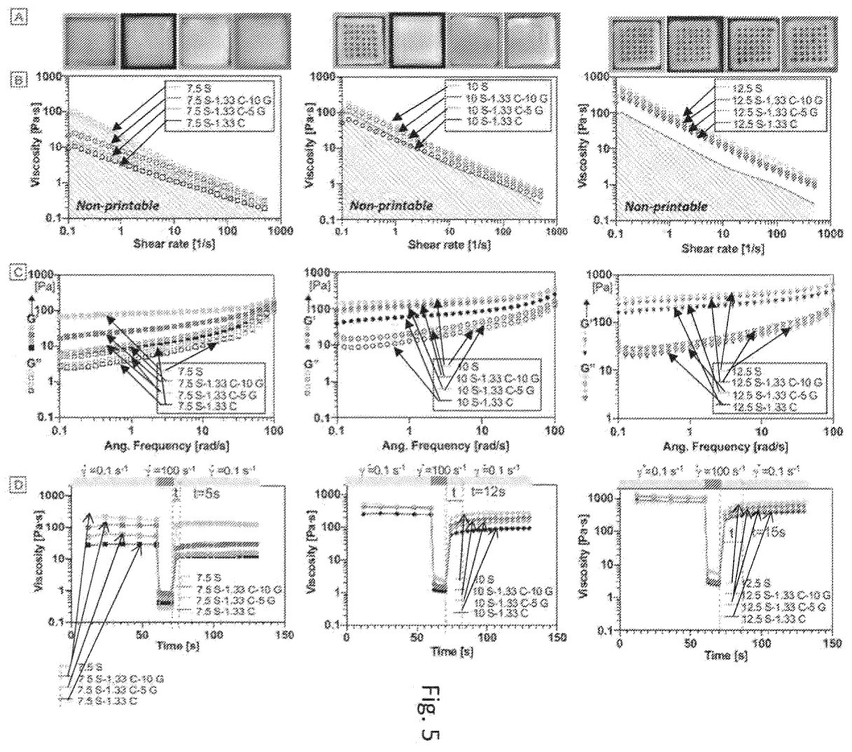

Starch is the major polysaccharide in plants and consists of a large number of repeated glucose units joined by α-D-(1-4) and/or α-D-(1-6) linkages. Starch has been recognized as a natural and biocompatible material in the field of tissue engineering due to its great biocompatibility, biodegradability, ultra-low-cost, non-toxicity, and appropriate pore size and morphology. Typically, the rheological property of starch is governed by the ratio of two structural components: amylose and amylopectin (Biduski et al., 2018). Generally, the amylose impairs the gel strength, and the amylopectin determines the gel viscosity. Normal corn starch contains 20%-30% amylose (X. Zhang et al., 2016). The formation of starch hydrogel is a three-step thermal treatment that includes swelling, gelatinization, and retrogradation to form a 3D hydrogel network (FIG. 4) (Wang et al., 2015).

To introduce the GNPs into the starch hydrogel, the GNPs were first suspended with the desired concentration into 50 mM HEPES buffer, and the GNP-supplemented starch hydrogel was formed by vigorous stirring of the starch granules in buffer for 20 min. Additionally, collagen was added as a cell-instructive component because collagen is the main component of most tissues and organs within the body, accounting for more than 30% of total protein mass. Collagen forms fibrous networks in the body, which can enhance the tissue structure and function of ECM while promoting cell adhesion, growth, tissue morphogenesis, and biological signaling. By varying the concentration of each component, 6 different nanocomposite hydrogels were prepared, as well as 6 control samples. The combinations were analyzed, and a combination was selected to print an anatomical ear structure to demonstrate the excellent printing fidelity, structural integrity, cell growth, and biocompatibility, as depicted in FIG. 3. The combination selected to print the ear contained 12.5% w/v starch, 1.33 mg/mL collagen, and 10×10⁹ gelatin nanoparticles. The printed scaffold also exhibited an improved biological property to promote cell attachment and proliferation, as shown in FIG. 3 by culturing NIH 3T3 cells for 7 days.

### Example 3: Rheological Characterization of Nanocomposite Hydrogels

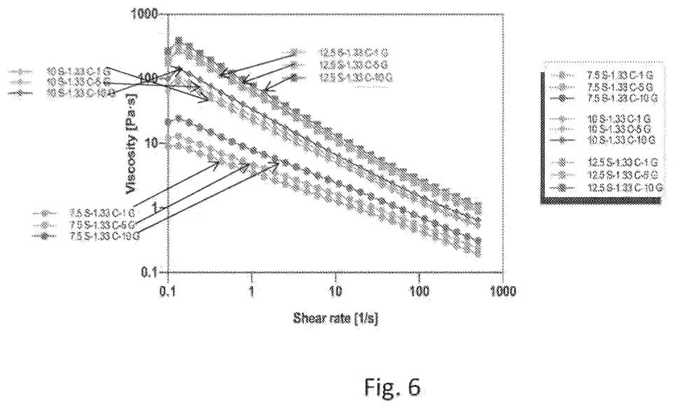

The rheological profiles of variable combinations of GNPs, starch hydrogel, and collagen were investigated, which could inform the development of high-quality bio-ink with fine printability and 3D shape. As shown in FIG. 5, by printing a 10-layer scaffold in a grid pattern, starch was observed to be the dominant factor to maintain the 3D printability, as long as the concentration was larger than 10%. It appeared that the addition of collagen adversely affected the printability. This observation was further quantitatively validated by shear rate sweep analysis in (b) in FIG. 5, along with the pure starch hydrogels and starch-collagen blend as the control group. A significant increase in viscosity was observed along with increasing starch concentration across all the groups. The addition of collagen resulted in a 90.52%, 61.51%, and 38.91% reduction of zero-shear viscosity, compared with the corresponding groups with 7.5%, 10%, and 12.5% pure starch hydrogel only, which indicates the reduced influence from collagen on the overall viscosity with increased starch concentration. The final concentration of collagen at 1.33 mg mL⁻¹ in the hydrogel system exhibited extremely low viscosity (˜5.3 mPa·s) which is close to water viscosity at low temperature, thus, leading to the reduced viscosity due to the dilution of the entire hydrogel system (B. Biduski et al. Int. J Biol. Macromol. 2018, 113, 443). However, along with the increased starch concentration overpowering the influence on the overall material, the collagen influence on viscosity reduction of the overall material was minimized. The incorporation of GNPs enhanced the viscosity of the starch-collagen blend in a concentration-dependent manner, as shown in FIG. 6. With a GNP concentration of 1×10¹⁰ particles/mL, the formed nanocomposite starch-collagen hydrogel exhibited reinforced viscosity, comparable to the pure starch, but with significantly enhanced cellular interaction sites introduced by the collagen.

Frequency sweeps offer a well-defined comparison of viscoelastic properties under constant strain. As shown in (inset (c) in FIG. 5), the storage modulus G′ was larger than the loss modulus G″ under the applied angular frequency in the entire frequency region for all groups. Additionally, both G′ and G″ increased with increasing angular frequency. Although no cross-over point appeared in any group, G″ and G′ of samples with a starch concentration at 7.5% and 10% converged at higher values of ω. These results imply the gradual loss in the elastic property in the tested gel-like materials. Overall, frequency sweep analysis cooperatively confirmed the significance of starch in maintaining the mechanical property of all the tested groups, as well as the beneficial effect from the presence of collagen and gelatin nanoparticles on manipulating the viscosity and mechanical strength of the nanocomposite hydrogel, making it tunable to specific applications.

Thixotropy is a time-dependent shear-thinning behavior that is considered to be associated with the rheological instability of a material. By applying a certain shear rate, the weak physical bonds are mechanically disrupted, and the inner structures disintegrate into separate aggregates. This process is shear rate- and time-dependent. Nevertheless, Brownian motion leading to collisions can restore aggregation upon the removal of the shear rate. Therefore, thixotropy plays a pivotal role in determining the shape fidelity of 3D printed constructs. The thixotropic properties of the nanocomposite hydrogel samples were investigated through a three-phase measurement. As shown in the (d) inset of FIG. 5, all of the tested samples exhibited thixotropic properties at different levels due to the varied components. At a given starch concentration, starch, starch-collagen blend, and the nanocomposite hydrogels showed similar recovery trends, in terms of time to reach equilibrium. Meanwhile, an increased starch concentration yielded a longer recovery time. For starch at varied concentrations of 7.5%, 10%, and 12.5%, the recovery time was 5 s, 12 s, and 15 s, respectively. With a starch concentration at 10% and 12.5%, introducing GNPs significantly improved the recovery performance in comparison to the starch-collagen blend, showing a comparable recovery rate to the pure starch hydrogel. For instance, the initial viscosities of 12.5 S-1.33 C-10 G and 12.5 S-1.33 C were ˜995 Pa·s and ˜763 Pa·s, and they immediately dropped to ˜3 Pa·s when increasing the shear rate to 100 s⁻¹. Upon the decrease of shear rate to 0.1 s⁻¹, the viscosities returned to 591 and 392 Pa·s, which were 59.5% and 51.5% of their initial viscosity, respectively.

The starch hydrogel was prepared at 45° C. Starch gelatinization is highly dependent on temperature, and the thixotropic property of starch is correlated with the degree of granule pasting (Sikora et al., 2015). Therefore, although it is generally accepted that the pregelatinized starch can generate instant viscosity in water at room temperature, the dissolving of the starch granules could be incomplete at the recommended temperature. Hence, the remaining non-melted starch granules may disperse within the structure formed by entangled amylopectin and amylose molecules. The presence of the swollen but non-melted starch granules could cause a retarded and incomplete recovery. Despite the relatively slow recovery, the nanocomposite hydrogel demonstrated shear-thinning and thixotropic properties, which are desirable for extrusion-based bioprinting.

### Example 4: Printability and Structural Integrity

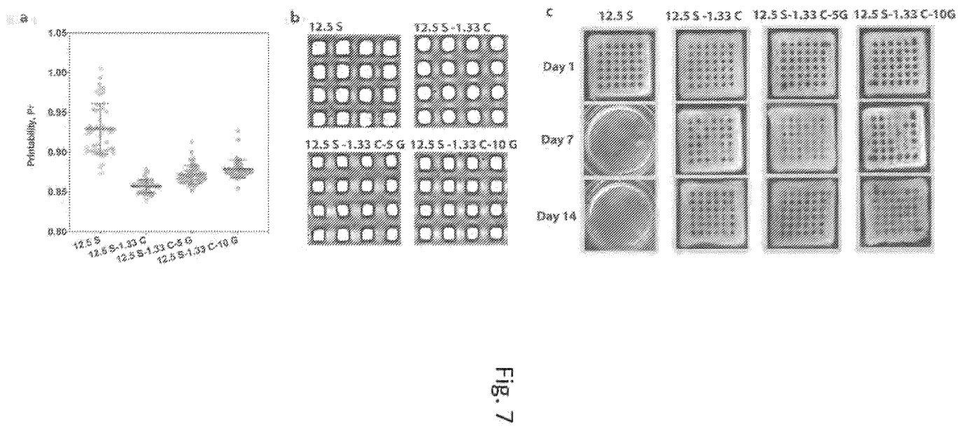

When creating a 3D bioprinted construct, the ability of the printed material to maintain the desired shape during and after printing is an important factor to consider. This desired shape can be influenced by intrinsic material properties and printing parameters, such as pressure and printing speed. The desired outcome for the extruded material is to have constant width and smooth edges in the shape of the extrusion path without bulging, thinning, or breaking. The material should have enough strength for self-support, as well as high shape fidelity. As reported in Ouyang et al., 2016, the printability of materials may be quantitatively investigated using the following equation:

$$P r = \frac{π}{4} \cdot \frac{1}{C} , where C = \frac{4 π A}{{L}^{2}} ,$$

where A is the area, and L is the perimeter of the enclosed area. Therefore, a circle has the highest circularity, equal to 1, while a square has a C value of π/4. The larger the Pr value is, the better the printability is, which may indicate less impairment on printing resolution and 3D stacking from reduced material viscosity and gelation degree. For an ideal gelation condition or perfect printability status, the interconnected channels of the constructs would demonstrate a square shape, and the Pr value would be 1. From an investigation, the results for which are shown in FIG. 5 (inset a), a Pr value larger than 0.8 could show acceptable printability. From published literature, a Pr value within the range of 0.9-1.1 demonstrated satisfactory filament morphology and mechanical stability of the printed construct (W. Lim et al. Polymers 2021, 13, 1773; L. Ouyang, R. Yao et al. Biofabrication 2016, 8, 035020). The printability of biomaterial inks with a starch concentration at 12.5% was calculated using the above Equation. As shown in FIG. 7, starch-collagen blends showed the circular enclosed area between the printed filaments, and thus a relatively lower Pr value (0.857±0.008). Pure starch had a highest Pr value at 0.929±0.032 and the thinnest filaments, demonstrating superior 3D printability (see Table 2). Enhanced shape fidelity was observed with the supplement of GNPs, which is in good agreement with the rheological measurement results.

TABLE 2
Printability value and filament
width of each combination hydrogel

| | | 12.5 [symbol] - | 12.5 [symbol] - | 12.5 [symbol] - |
|---|---|---|---|---|
| | 12.5 [symbol] | 1.33C | 1.33C-5G | 1.33C-10G |
| P [symbol] | 0.929 ± 0.032 | 0.857 ± 0.008 | 0.872 ± 0.012 | 0.879 ± 0.011 |
| Filament | 0.814 ± 0.041 | 0.952 ± 0.037 | 1.147 ± 0.023 | 0.92 ± 0.017 |
| width | | | | |

[symbol]

indicates data missing or illegible when filed

An ideal scaffold is required to support long term cell culture. Therefore, aside from the printability, the structural integrity and stability of the scaffold during culture should be considered. Inset (C) in FIG. 7 shows the representative images of all the tested samples over a 14-day immersion in PBS. Notably, the printed structure using 12.5% starch hydrogel was dispersed within 24 hours of culture and 3D structures were corrupted, whereas the other samples were displaying more robust and stable structures. This fast disintegration of pure starch could be attributed to incomplete gelatinization of starch granules. Conversely, the thermo-gelation of collagen took place during culture, which could further stabilize the constructs for supporting long-term 3D cell culture and tissue growth.

### Example 5: Morphological Microstructure Characterization

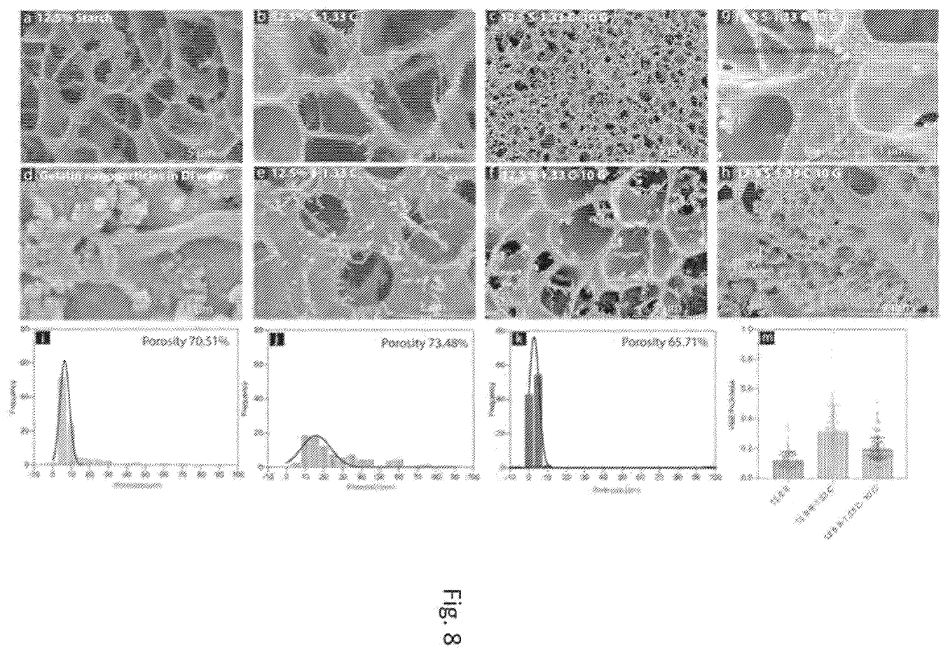

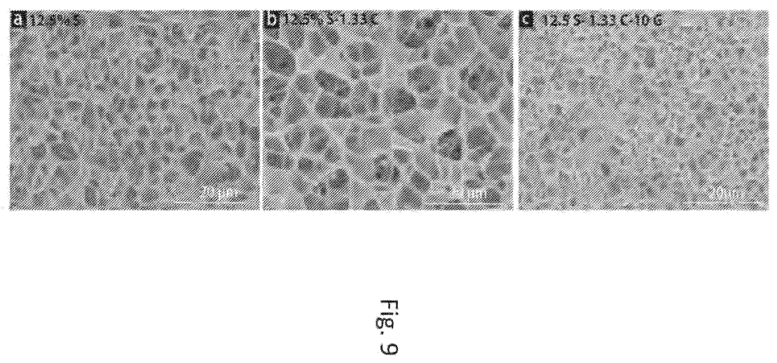

The porosity plays an important role in scaffolding biomaterials, which can facilitate mass transport associated nutrient and biological exchange for promoting 3D cell growth. The microstructures of starch hydrogel, starch-collagen blend, and nanocomposite hydrogel with a fixed starch concentration at 12.5% were examined using a cryo-scanning electron microscope (Cryo-SEM). ImageJ was used to analyze the Cryo-SEM images to determine the pore size distribution, porosity, and wall thickness within the hydrogel matrix. GNPs in DI water were also imaged as a control (FIG. 8). As shown in FIG. 9, the interconnected hierarchical porous structure was observed in all tested hydrogel samples at a lower magnification. This honeycomb-like porous structure implies the capacity to facilitate cell migration, proliferation, oxygen, and nutrient transport, as well as potential applications in drug loading and delivery in the void space.

Inset (a) in FIG. 8 shows that the pure starch hydrogel possesses a smooth surface and relatively thin walls throughout the entire matrix, along with some starch fibers stretching out from the surface. Adding collagen to starch brought heterogeneity to the pore surface, as seen in inset (b) in FIG. 8. The overall microstructure of the starch-collagen blend is easily identifiable, with much larger hierarchical pores and collagen fibers randomly distributed on the pore surface of the starch. Notably, the addition of collagen gel significantly thickened the wall of the blend hydrogel in comparison to that in starch. The wall thickness in the starch-collagen blend was 0.30±0.15 μm, which is 2-fold that in pure starch hydrogel (0.13±0.06 μm). Similarly, in nanocomposite hydrogel, collagen still maintained a distinctive fibrous structure as what was observed previously in the starch-collagen blend (FIG. 8, insets (c), (f), and (h)). The introduction of GNPs gave rise to a higher homogeneous microporous structure and evenly distributed collagen fibers; furthermore, the nanocomposite hydrogel displayed a largely compacted packing of hydrogel networks. A smaller average pore size than that of the control groups was observed as a result of the additional GNPs. As illustrated in (g) of FIG. 8, some of the loaded GNPs merged with the skeleton of the matrix to strengthen the entire hydrogel networks Additionally, given the intermolecular interactions between GNPs and collagen fibers, some of the GNPs were recognizably bonded to the collagen fibers, connecting with them and forming an interlaced or weblike structure ((f) in FIG. 8). Each of (i), (j), and (k) in FIG. 8 displays the histogram of pore size distribution and porosity of all the samples. Starch-collagen blend with a highly porous structure shows a much broader pore size distribution and highest porosity at 73.48% than the rest of the tested samples, highlighting the loose structure of the matrix. With the introduction of GNPs, the pores were more uniformly distributed and much smaller in size. The results further validate the indispensable role of GNPs in reinforcing the mechanical stiffness of the nanocomposite hydrogel.

### Example 6: In Vitro Cell Culture on 3D Printed Nanocomposite Starch-Based Hydrogel Scaffold

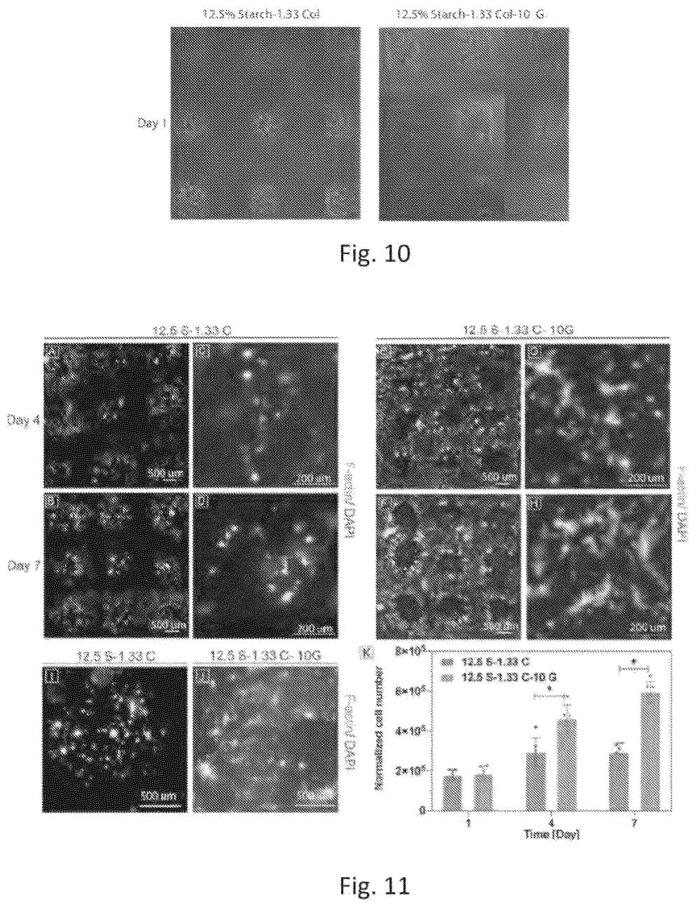

Combining the rheological characterization and microstructure evaluation, the nanocomposite hydrogel offers a desirable mechanical and geometrical environment. In assessing the biocompatibility of the nanocomposite hydrogel, NIH 3T3 fibroblasts were seeded on top of the 3D printed scaffolds with the selected biomaterial ink. Since the pure starch scaffolds experience a rapid collapse during culture, which is not conducive for long-term culture, the starch-collagen blend was printed in parallel as a control. FIG. 10 shows the cell distribution after seeding for 3 h at day 1 with consistent seeding density across all samples (seeding density ˜106 cells/mL). The majority of the cells were resting in the cavity of printed grid pattern. The cellular response was observed for 7 days. As shown in FIG. 11, vigorous cell migration and spreading were observed on day 4 for cells seeded on the nanocomposite scaffolds ((a) and (b) in FIG. 11, whereas the cells seeded on the starch-collagen blend scaffolds were visualized with limited cell spreading (Figure (c) and (d) in FIG. 11). On day 7, cells were more uniformly distributed on the nanocomposite scaffold with robust elongation and network formation ((g) and (h) in FIG. 11). FIG. 11 (j) presents another view of cell distribution and elongation on the printed filament. In contrast, cells on starch-collagen scaffolds were mainly stuck in the printed cavities of the scaffold, displaying sparse cell elongation and spreading ((e) and (f) in FIG. 11). Cell proliferation shown in (k) in FIG. 11 demonstrates a faster cell proliferation on nanocomposite scaffolds with a nearly 2-fold enhancement in the nanocomposite starch hydrogel bio-ink described herein. This indicates that the loaded GNPs dramatically improved the biological property. Collectively, the results highlight the enhancement on cell attachment, elongation, and migration by using a developed material blend, which could be ideal 3D tissue scaffolding for improving 3D cell growth.

### CONCLUSION

Starch is an appealing natural biopolymer for versatile tissue engineering applications, owing to its cost-effectiveness, scalable production, biocompatibility, and biodegradability. However, because of its mechanical instability and absence of cell-binding sites, starch has not been widely used as a 3D bio-ink in the field of tissue scaffolding. As shown herein, a 3D printable starch-based hydrogel with desirable biological properties and improved stability was realized by blending starch with collagen and gelatin nanoparticles. Through rheological characterization, this nanocomposite starch hydrogel exhibited highly desirable shear-thinning and thixotropic properties, as well as mechanical strength suitable for high-fidelity 3D printing. Gelatin nanoparticles also functioned as a rheological modifier, which could tune the mechanical strength over a wide range for various tissue engineering applications. By formulating starch with gelatin nanoparticles, the resulting bio-ink showed significantly enhanced mechanical properties, which preserved shape fidelity, and compensated the loss of mechanical strength when blended with collagen. The unique homogeneous microporous structure with abundant collagen fibers and GNP interlaced web-like structure not only supports efficient mass transport, but also supplies rich attachment sites for promoting 3D cell growth, as evidenced by culturing fibroblast cells with fold increased proliferation rate. Due to their enhanced mechanical strength, the developed starch nanocomposite hydrogel scaffolds can also maintain good 3D structural integrity with a reduced degradation rate by being gradually replaced along with cell growth. This was modeled to match the complexity of the real tissue microenvironment, which is critically needed for long-term tissue culture.

Although there are many developed scaffold biomaterials in the field of tissue engineering and regenerative medicine, clinical translation is still hampered due to toxicity and biocompatibility concerns. The chemical and mechanical properties of the biomaterial scaffold must be optimized to suit the interaction with cells and the surrounding tissue microenvironment, including the efficient mass transport and host tissue integration. The nanocomposite starch bio-ink materials described herein can have their mechanical strength tuned by varying the concentration of each component that the materials contain. This offers a great opportunity to tailor the material to the specific tissue environment. Additionally, the developed nanocomposite starch is non-toxic and non-chemically modified; therefore, the starch nanocomposite is still highly biodegradable by converting carbohydrates back into forms that are usable for various biosynthetic and metabolic routes in vivo.

### REFERENCES

[1] M. Askari, M. Afzali Naniz, M. Kouhi, A. Saberi, A. Zolfagharian, M. Bodaghi, Biomater. Sci. 2021, 9, 535.
[2] N. Liu, S. Huang, B. Yao, J. Xie, X. Wu, X. Fu, Sci. Rep. 2016, 6, 34410.
[3] P. Zhuang, J. An, C. K. Chua, L. P. Tan, Mater. Des. 2020, 193, 108794.
[4] R. Attalla, E. Puersten, N. Jain, P. R. Selvaganapathy, Biofabrication 2018, 11, 15012.
[5] J. Idaszek, M. Costantini, T. A. Karlsen, J. Jaroszewicz, C. Colosi, S. Testa, E. Fornetti, S. Bernardini, M. Seta, K. Kasarello, R. Wrzesien, S. Cannata, A. Barbetta, C. Gargioli, J. E. Brinchman, W. Świeszkowski, Biofabrication 2019, 11, 44101.
[6] J. M. Unagolla, A. C. Jayasuriya, Appl. Mater. Today 2020, 18, 100479.
[7] P. Zhuang, A. X. Sun, J. An, C. K. Chua, S. Y. Chew, Biomaterials 2018, 154, 113.
[8] A. Schwab, R. Levato, M. D'Este, S. Piluso, D. Eglin, J. Malda, Chem. Rev. 2020, 120, 11028.
[9] Y. S. Zhang, A. Khademhosseini, Science 2017, 356, eaaf3627.
[10] E. O. Osidak, V. I. Kozhukhov, M. S. Osidak, S. P. Domogatsky, Int. J. Bioprint. 2020, 6, 270.
[11] P. Zhuang, W. L. Ng, J. An, C. K. Chua, L. P. Tan, PLoS One 2019, 14, e0216776.
[12] S. Zhang, D. Huang, H. Lin, Y. Xiao, X. Zhang, Biomacromolecules 2020, 21, 2400.
[13] L. Ouyang, J. P. K. Armstrong, Y. Lin, J. P. Wojciechowski, C. Lee-Reeves, D. Hachim, K. Zhou, J. A. Burdick, M. M. Stevens, Sci. Adv. 2020, 6, eabc5529.
[14] H. Chen, F. Xie, L. Chen, B. Zheng, J. Food Eng. 2019, 244, 150.
[15] J. J. Perez, N. J. Francois, G. A. Maroniche, M. P. Borrajo, M. A. Pereyra, C. M. Creus, Carbohydr. Polym. 2018, 202, 409.
[16] B. Amal, B. Veena, V. P. Jayachandran, J. Shilpa, J. Mater. Sci.: Mater. Med. 2015, 26, 181.
[17] R. Shi, A. Zhu, D. Chen, X. Jiang, X. Xu, L. Zhang, W. Tian, J. Appl. Polym. Sci. 2010, 115, 346.
[18] X. Wen, M. Shen, Y. Bai, C. Xu, X. Han, H. Yang, L. Yang, J. Biomed. Mater. Res., Part B 2020, 108, 104.
[19] D. Wu, A. Samanta, R. K. Srivastava, M. Hakkarainen, Biomacromolecules 2017, 18, 1582.
[20] F. Mirab, M. Eslamian, R. Bagheri, Biomed. Phys. Eng. Express 2018, 4, 55021.
[21] A. Eskandarinia, A. Kefayat, M. Rafienia, M. Agheb, S. Navid, K. Ebrahimpour, Carbohydr. Polym. 2019, 216, 25.
[22] F. G. Torres, S. Commeaux, O. P. Troncoso, Starch/Staerke 2013, 65, 543.
[23] Y. Mao, M. Pan, H. Yang, X. Lin, L. Yang, Front. Mater. Sci. 2020, 14, 232.
[24] V. S. Waghmare, P. R. Wadke, S. Dyawanapelly, A. Deshpande, R. Jain, P. Dandekar, Bioact. Mater. 2018, 3, 255.
[25] H. M. Butler, E. Naseri, D. S. MacDonald, R. Andrew Tasker, A. Ahmadi, Materialia 2020, 12, 100737.
[26] A. Nadernezhad, O. S. Caliskan, F. Topuz, F. Afghah, B. Erman, B. Koc, ACS Appl. Bio Mater. 2019, 2, 796.
[27] S. A. Wilson, L. M. Cross, C. W. Peak, A. K. Gaharwar, ACS Appl. Mater. Interfaces 2017, 9, 43449.
[28] S. Piluso, M. Labet, C. Zhou, J. W. Seo, W. Thielemans, J. Patterson, Biomacromolecules 2019, 20, 3819.
[29] B. Begines, A. Alcudia, R. Aguilera-Velazquez, G. Martinez, Y. He, G. F. Trindade, R. Wildman, M.-J. Sayagues, A. Jimenez-Ruiz, R. Prado-Gotor, Sci. Rep. 2019, 9, 16097.
[30] K. Behera, Y. H. Chang, M. Yadav, F. C. Chiu, Polymer 2020, 186, 122002.
[31] Z. Cheng, L. Xigong, D. Weiyi, H. Jingen, W. Shuo, L. Xiangjin, W. Junsong, J Nanobiotechnol. 2020, 18, 97.
[32] S. Ruan, X. Cao, X. Cun, G. Hu, Y. Zhou, Y. Zhang, L. Lu, Q. He, H. Gao, Biomaterials 2015, 60, 100.
[33] B. Biduski, W. M. F. d. Silva, R. Colussi, S. L. d. M. E. Halal, L. T. Lim, Á. R. G. Dias, E. d. R. Zavareze, Int. J. Biol. Macromol. 2018, 113, 443.
[34] S. Wang, C. Li, L. Copeland, Q. Niu, S. Wang, Compr. Rev. Food Sci. Food Saf 2015, 14, 568.
[35] J. A. Paten, S. M. Siadat, M. E. Susilo, E. N. Ismail, J. L. Stoner, J. P. Rothstein, J. W. Ruberti, ACS Nano 2016, 10, 5027.
[36] L. Ouyang, R. Yao, Y. Zhao, W. Sun, Biofabrication 2016, 8, 35020.
[37] W. Lim, S. Y. Shin, J. M. Cha, H. Bae, Polymers 2021, 13, 1773.
[38] L. Ouyang, R. Yao, Y. Zhao, W. Sun, Biofabrication 2016, 8, 035020.
[39] C. J. Coester, K. Langer, H. van Briesen, J. Kreuter, J. Microencapsulation 2000, 17, 187.
[40] B. Burkel, B. A. Morris, S. M. Ponik, K. M. Riching, K. W. Eliceiri, P. J. Keely, J. Visualized Exp. 2016, 111, 53989.
[41] S. K E, S. G, P. C, T. SM, E. KW, K. P J, F. A, B. DJ, Biomaterials 2009, 30, 4833.

Efforts have been made to ensure accuracy with respect to numbers used (e.g., amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

1. A nanocomposite hydrogel composition for three-dimensional printing, the nanocomposite hydrogel composition comprising collagen, starch, and gelatin nanoparticles.

2. The nanocomposite hydrogel composition of claim 1, wherein the collagen is present in the nanocomposite hydrogel composition at a concentration of about 0.1 mg/mL to about 15 mg/mL.

3. The nanocomposite hydrogel composition of claim 2, wherein the collagen is present in the nanocomposite hydrogel composition at a concentration of about 1 mg/mL to about 2 mg/mL.

4. The nanocomposite hydrogel composition of claim 3, wherein the collagen is present in the nanocomposite hydrogel composition at a concentration of about 1.33 mg/mL.

5. The nanocomposite hydrogel composition of claim 1, wherein the starch is present in the nanocomposite hydrogel composition at a concentration of about 2% w/v to about 25% w/v.

6. The nanocomposite hydrogel composition of claim 5, wherein the starch is present in the nanocomposite hydrogel composition at a concentration of about 7% w/v to about 15% w/v.

7. The nanocomposite hydrogel composition of claim 6, wherein the starch is present in the nanocomposite hydrogel composition at a concentration of about 12.5% w/v.

8. The nanocomposite hydrogel composition of claim 1, wherein the gelatin nanoparticles are present in the nanocomposite hydrogel composition at a concentration of about 1*10⁷ particles/mL to about 10*10¹³ particles/mL.

9. The nanocomposite hydrogel composition of claim 8, wherein the gelatin nanoparticles are present in the nanocomposite hydrogel composition at a concentration of about 3*10⁹ particles/mL to about 12*10⁹ particles/mL.

10. The nanocomposite hydrogel composition of claim 1, wherein the composition comprises:
starch at a concentration of about 7.5% w/v, about 10% w/v, or about 12.5% w/v,
collagen at a concentration of about 1.33 mg/mL, and
gelatin nanoparticles at a concentration of about 5*10⁹ particles/mL or about 10*10⁹ particles/mL.

11. The nanocomposite hydrogel composition of claim 10, wherein the composition comprises:
starch at a concentration of about 12.5% w/v,
collagen at a concentration of about 1.33 mg/mL, and
gelatin nanoparticles at a concentration of about 10*10⁹ particles/mL.

12. The nanocomposite hydrogel composition of claim 1, wherein the gelatin nanoparticles have a peak particle size of about 1 nm to about 500 nm as determined by nanoparticle tracking analysis (NTA).

13. The nanocomposite hydrogel composition of claim 12, wherein the gelatin nanoparticles have a peak particle size of about 40 nm as determined by NTA.

14. The nanocomposite hydrogel composition of claim 1, wherein the nanocomposite hydrogel composition is biodegradable.

15. The nanocomposite hydrogel composition of claim 1, wherein the nanocomposite hydrogel composition is biocompatible.

16. The nanocomposite hydrogel composition of claim 1, wherein the nanocomposite hydrogel composition exhibits at least one of the following properties: printability, shear-thinning, shape fidelity, mechanical strength, or thixotropy.

17. The nanocomposite hydrogel composition of claim 1, wherein the nanocomposite hydrogel composition is characterized by an interconnected, dense porous structure.

18-24. (canceled)

25. A method of treating a wound and/or regenerating tissue in a subject in need thereof, the method comprising:
administering to a treatment site in the subject a nanocomposite hydrogel composition comprising collagen, starch, and gelatin nanoparticles.

26. The method of claim 25, wherein the nanocomposite hydrogel composition is printed at the treatment site to produce a three-dimensional object.

27. The method of claim 25, wherein the treatment site is on an external surface of the subject.

28. The method of claim 25, wherein the treatment site is at an internal location within the subject.

29. A method of preparing a nanocomposite hydrogel composition, the method comprising:
contacting a mass of starch with a mixture comprising a mass of gelatin nanoparticles and a volume of water to form a first mixture;
cooling the first mixture; and,
contacting the first mixture with a mass of collagen, thereby forming the nanocomposite hydrogel composition,
wherein the method for preparing the nanocomposite hydrogel composition does not comprise chemical cross-linking.

30. The method of claim 29, wherein said contacting starch with the mixture comprising the mass of gelatin nanoparticles and the volume of water is maintained during the preparing the nanocomposite hydrogel composition at a temperature of between about 0° C. and about 100° C.

31. The method of claim 29, wherein the contacting the mass of starch with the mixture comprising the mass of gelatin nanoparticles and the volume of water proceeds for a time of between about 15 minutes to about 60 minutes.

32. The method of claim 29, wherein the cooling the first mixture is carried out at a temperature of about 4° C.

33. The method of claim 29, wherein the cooling the first mixture proceeds for a time of between about 1 hour and about 4 hours.

34. The method of claim 29, wherein the contacting the first mixture with the mass of collagen proceeds for a time of between about 30 minutes and about 6 hours.
